(19) United States
(12) Patent Application Publication
(10) Pub. No.: US 2023/0395248 A1
(43) Pub. Date: Dec. 7, 2023

(54) Determination of Cardiac Condition Measure based on Machine Learning Analysis of ECG and/or Cardio-vibrational Data

(71) Applicant: ZOLL Medical Corporation, Chelmsford, MA (US)

(72) Inventors: Michael Sipe, Pittsburgh, PA (US); Shinya Matsui, Pittsburgh, PA (US)

(73) Assignee: ZOLL Medical Corporation, Chelmsford, MA (US)

(21) Appl. No.: 18/033,291

(22) PCT Filed: Oct. 25, 2021
(86) PCT No.: PCT/US2021/056488

§ 371 Date: Apr. 21, 2023

Related U.S. Application Data
(60) Provisional application No. 63/106,144, filed on Oct. 27, 2020.

(51) Int. Cl. G16H 40/63; G06N 3/045; G06N 3/08; G06N 3/0464

(52) U.S. Cl. CPC G16H 40/63; G06N 3/045; G06N 3/08; G06N 3/0464

(57) ABSTRACT

Data samples derived from raw physiological data captured by sensors of a wearable medical device monitoring a patient's heart, such as a first ECG channel data sample, a second ECG channel data sample, and/or a cardio-vibrational data sample, are applied to a multi-tier machine learning engine to determine a cardiac condition measure traditionally calculated based upon information typically derived in a laboratory or clinical setting. A first tier of the multi-tier machine learning engine may analyze the physiological data sample(s) using first machine learning classifier(s) to obtain a first result. The first result may then be applied to second machine learning classifier(s) of a second tier of the machine learning engine, along with physiological metrics and/or patient clinical information, to determine the cardiac condition measure.

### RELATED APPLICATIONS

This application is a national stage application of international application No. PCT/US2021/056488 entitled “Determination of Cardiac Condition Measure Based on Machine Learning Analysis of ECG and/or Cardio-Vibrational Data,” which claims priority to U.S. Provisional Patent Application Ser. No. 63/106,144, entitled “Determination of Cardiac Condition Measure based on Machine Learning Analysis of ECG and/or Cardio-Vibrational Data,” filed Oct. 27, 2020. All above identified applications are hereby incorporated by reference in their entireties.

### BACKGROUND

Some conventional medical devices that monitor the cardiopulmonary system obtain a subject's electrocardiogram (ECG) signal from body surface electrodes. Known ambulatory wearable defibrillators, such as the LifeVest® Wearable Cardioverter Defibrillator available from ZOLL Medical Corporation of Chelmsford, Mass., use four ECG sensing electrodes in a dual-channel bipolar configuration. This arrangement of ECG sensing electrodes is usually suitable because in most cases it is rare that noise or electrode movement affects the entire body circumference. The dual-channel bipolar configuration provides redundancy and allows the system to operate on a single channel if necessary. Because signal quality also varies from subject to subject, having two channels provides the opportunity to have improved signal pickup, since the ECG sensing electrodes are located in different body positions. Heart rhythms may also be monitored using vibrational sensors (e.g., including acoustic sensors and/or audio transducers) to detect and record cardio-vibrational signals and the timing of the cardio vibrations, including any one or all of S1, S2, S3, and S4 cardio vibrations.

### SUMMARY OF ILLUSTRATIVE EMBODIMENTS

In one aspect, the present disclosure relates to a method for determining a cardiac condition measure of a patient wearing a wearable medical device based on machine learning analysis, the method including obtaining, by processing circuitry from the wearable medical device, physiological data representing a sample timeframe, the physiological data including ECG data representing ECG signals of a patient, where the ECG signals were collected by at least two ECG electrodes of the wearable medical device monitoring a heart of the patient, and/or cardio-vibrational data representing cardio-vibrational signals of the patient, where the cardio-vibrational signals were collected by at least one vibrational sensor of the wearable medical device monitoring the heart of the patient. The method may include applying, by the processing circuitry, the physiological data to a machine learning engine to determine the cardiac condition measure of the patient, where applying includes applying the ECG data and/or the cardio-vibrational data to a first tier of the machine learning engine including one or more first machine learning classifiers to obtain a first result, and applying the first result along with i) clinical information regarding the patient and/or ii) physiological metrics determined from signals collected by the wearable medical device to a second tier of the machine learning engine including one or more second machine learning classifiers to obtain a second result. The method may include determining, by the processing circuitry at least in part from the second result, the cardiac condition measure.

In some embodiments, the second tier includes one or more fully connected artificial neural network layers. The first tier may include one or more convolutional artificial neural network layers. In some embodiments, i) the one or more first machine learning classifiers include at least two stages of machine learning classifiers of the first tier and/or ii) the one or more second machine learning classifiers include at least two stages of machine learning classifiers of the second tier.

In some embodiments, the one or more first machine learning classifiers include an ECG trained machine learning classifier and a cardio-vibrational data trained machine learning classifier. The one or more first machine learning classifiers may be configured to implement sets of convolutional filters, each being sized between about 2 and 10 weights, between about 10 and 50 weights, between about 50 and 100 weights, or between about 100 and 10000 weights. The one or more first machine learning classifiers may be tuned using a predetermined number of filters, where the predetermined number of filters is less than 4, between 4 and 6, between 6 and 10, or up to 15.

In some embodiments, the first result includes features extracted from the physiological data. The cardiac condition measure may be one of Class I heart failure (HF), Class II HF, Class III HF, or Class IV HF in accordance with the New York Heart Association (NYHA) classification system. The cardiac condition measure may be an ejection fraction measure ranging from 0% to 100%. The cardiac condition measure may be an ejection fraction classification. The ejection fraction classification may include one of a) an ejection fraction of greater than 30% as a first ejection fraction classification, and an ejection fraction of less than 30% as a second ejection fraction classification, b) an ejection fraction of greater than 35% as the first ejection fraction classification, and an ejection fraction of less than 35% as the second ejection fraction classification, c) an ejection fraction of greater than 40% as the first ejection fraction classification, and an ejection fraction of less than 40% as the second ejection fraction classification, or d) an ejection fraction of greater than 45% as the first ejection fraction classification, and an ejection fraction of less than 45% as the second ejection fraction classification.

In some embodiments, the method includes providing, by the processing circuitry, information regarding the cardiac condition measure to a remote computing device for review by a medical professional, clinician, or caregiver. The method may include providing, by the processing circuitry in real time for review by the patient, a medical professional, clinician, or caregiver, information corresponding to the cardiac condition measure via an output device of the wearable medical device or a separate computing device. Providing the information corresponding to the cardiac condition measure may include adding, for review within a medical professional patient information portal, a visual indication of worsened cardiac condition associated with the patient. The method may include providing, for review within the medical professional patient information portal, a visual display representing at least a portion of the physiological metrics.

In some embodiments, the sample timeframe is between about 10 seconds to about 20 seconds, between about 20 seconds to about 30 seconds, between about 30 seconds and about 40 seconds, between about 40 seconds and about 50 seconds, or between about 50 seconds and about 60 seconds. The physiological metrics may include one or more of electromechanical activation time (EMAT), time-corrected EMAT (EMATc), left ventricular systolic time (LVST), S3 intensity, S4 intensity, S3 duration, S4 duration, systolic dysfunction index (SDI), or heart rate. The clinical information may include one or more of an age of the patient, a gender of the patient, an indication of a coronary artery bypass grafting (CABG) procedure on the patient, an indication of a congenital heart defect (CONG) diagnosis of the patient, an indication of an implantable cardioverter defibrillator explant (EXPLANT) procedure on the patient, an indication of heart failure or cardiomyopathy (HFCM) in the patient, an indication of diagnosis of a prior myocardial infarction of the patient, an indication of prior ventricular fibrillation of the patient, an indication of prior ventricular tachycardia of the patient, or an indication of diagnosis of cardiac ischemia of the patient. The ECG data and/or the cardio-vibrational data may include raw signal data collected by the wearable medical device.

In some embodiments, the method includes accessing, by the processing circuitry, the clinical information from a non-transitory computer readable storage region. The method may include receiving, from the wearable medical device, at least a portion of the physiological metrics. The physiological data representing the sample timeframe may be obtained from a period of at least a) one second prior to applying the physiological data to the machine learning engine, b) one hour prior to applying the physiological data to the machine learning engine, c) 24 hours to applying the physiological data to the machine learning engine, or d) 3 days prior to applying the physiological data to the machine learning engine.

In some embodiments, the ECG data includes a front-to-back ECG data signal and a side-to-side ECG signal. The one or more first machine learning classifiers may include a front-to-back ECG trained machine learning classifier and a side-to-side ECG trained machine learning classifier.

In some embodiments, the method includes calculating, by the processing circuitry from at least one of the ECG data and the cardio-vibrational data, at least a portion of the physiological metrics. The method may include comparing, by the processing circuitry, at least one historic cardiac condition measure of the patient to the cardiac condition measure to identify a change in cardiac condition. The method may include receiving, from the wearable medical device, the at least one historic cardiac condition measure. Comparing the at least one historic cardiac condition measure of the patient to the cardiac condition measure may include calculating an improvement score quantifying a difference between the at least one historic cardiac condition measure and the cardiac condition measure. The at least one historic cardiac condition measure may be a previously determined cardiac condition measure determined by applying the physiological data to the machine learning engine.

In some embodiments, the wearable medical device is a hotter monitor. The wearable medical device may include a wearable cardioverter defibrillator. The wearable medical device may be configured to collect a 12 channel ECG signal.

In some embodiments, an area under the curve (AUC) performance of the machine learning engine is at least 70%, between about 70% and about 75%, between about 75% and about 80%, or between about 80% and about 85%, or between about 85% and about 95%, or between about 95% and about 99% when classifying the approximation of the cardiac condition measure into at least two classifications.

In one aspect, the present disclosure relates to a system for determining a cardiac condition measure of a patient wearing a wearable medical device based on machine learning analysis, the system including a non-volatile computer readable storage medium including physiological data representing a sample timeframe, the physiological data including ECG data representing ECG signals of the patient, where the ECG signals were collected by at least two ECG electrodes of the wearable medical device monitoring a heart of the patient, and/or cardio-vibrational data representing cardio-vibrational signals of the patient, where the cardio-vibrational signals were collected by at least one vibrational sensor of the wearable medical device monitoring the heart of the patient, and operations stored as computer executable instructions to a non-transitory computer readable media and/or encoded in hardware logic. The operations may be configured to apply the physiological data to a machine learning engine to determine the cardiac condition measure of the patient, where applying includes applying the ECG data and/or the cardio-vibrational data to a first tier of the machine learning engine including one or more first machine learning classifiers to obtain a first result, and applying the first result along with i) clinical information regarding the patient and/or ii) physiological metrics determined from signals collected by the wearable medical device to a second tier of the machine learning engine including one or more second machine learning classifiers to obtain a second result. The operations may be configured to determine, at least in part from the second result, the cardiac condition measure.

In some embodiments, the second tier may include one or more fully connected artificial neural network layers. The first tier may include one or more convolutional artificial neural network layers.

In some embodiments, i) the one or more first machine learning classifiers include at least two stages of machine learning classifiers of the first tier and/or ii) the one or more second machine learning classifiers include at least two stages of machine learning classifiers of the second tier. The one or more first machine learning classifiers may include an ECG trained machine learning classifier and a cardio-vibrational data trained machine learning classifier. The one or more first machine learning classifiers may be configured to implement sets of convolutional filters, each being sized between about 2 and 10 weights, between about 10 and 50 weights, between about 50 and 100 weights, or between about 100 and 10000 weights. The one or more first machine learning classifiers may be tuned using a predetermined number of filters, where the predetermined number of filters is less than 4, between 4 and 6, between 6 and 10, or up to 15.

In some embodiments, the first result includes features extracted from the physiological data. The cardiac condition measure may be one of Class I heart failure (HF), Class II HF, Class III HF, or Class IV HF in accordance with the New York Heart Association (NYHA) classification system. The cardiac condition measure may be an ejection fraction measure ranging from 0% to 100%. The cardiac condition measure may be an ejection fraction classification. The ejection fraction classification may include one of a) an ejection fraction of greater than 30% as a first ejection fraction classification, and an ejection fraction of less than 30% as a second ejection fraction classification, b) an ejection fraction of greater than 35% as the first ejection fraction classification, and an ejection fraction of less than 35% as the second ejection fraction classification, c) an ejection fraction of greater than 40% as the first ejection fraction classification, and an ejection fraction of less than 40% as the second ejection fraction classification, or d) an ejection fraction of greater than 45% as the first ejection fraction classification, and an ejection fraction of less than 45% as the second ejection fraction classification.

In some embodiments, the operations are configured to provide information regarding the cardiac condition measure to a remote computing device for review by a medical professional, clinician, or caregiver. The operations may be configured to provide, in real time for review by the patient, a medical professional, clinician, or caregiver, information corresponding to the cardiac condition measure via an output device of the wearable medical device or a separate computing device. Providing the information corresponding to the cardiac condition measure may include adding, for review within a medical professional patient information portal, a visual indication of worsened cardiac condition associated with the patient. The operations may be configured to provide, for review within the medical professional patient information portal, a visual display representing at least a portion of the physiological metrics.

In some embodiments, the sample timeframe is between about 10 seconds to about 20 seconds, between about 20 seconds to about 30 seconds, between about 30 seconds and about 40 seconds, between about 40 seconds and about 50 seconds, or between about 50 seconds and about 60 seconds. The physiological metrics may include one or more of electromechanical activation time (EMAT), time-corrected EMAT (EMATc), left ventricular systolic time (LVST), S3 intensity, S4 intensity, S3 duration, S4 duration, systolic dysfunction index (SDI), or heart rate. The clinical information may include one or more of an age of the patient, a gender of the patient, an indication of a coronary artery bypass grafting (CABG) procedure on the patient, an indication of a congenital heart defect (CONG) diagnosis of the patient, an indication of an implantable cardioverter defibrillator explant (EXPLANT) procedure on the patient, an indication of heart failure or cardiomyopathy (HFCM) in the patient, an indication of diagnosis of a prior myocardial infarction of the patient, an indication of prior ventricular fibrillation of the patient, an indication of prior ventricular tachycardia of the patient, or an indication of diagnosis of cardiac ischemia of the patient.

In some embodiments, the ECG data and/or the cardio-vibrational data includes raw signal data collected by the wearable medical device. The operations may be configured to access the clinical information from a non-transitory computer readable storage region. The operations may be configured to receive, from the wearable medical device, at least a portion of the physiological metrics. The physiological data representing the sample timeframe may be obtained from a period of at least a) one second prior to applying the physiological data to the machine learning engine, b) one hour prior to applying the physiological data to the machine learning engine, c) 24 hours to applying the physiological data to the machine learning engine, or d) 3 days prior to applying the physiological data to the machine learning engine.

In some embodiments, the ECG data includes a front-to-back ECG data signal and a side-to-side ECG signal. The one or more first machine learning classifiers may include a front-to-back ECG trained machine learning classifier and a side-to-side ECG trained machine learning classifier. The operations may be configured to calculate, from at least one of the ECG data and the cardio-vibrational data, at least a portion of the physiological metrics. The operations may be configured to compare at least one historic cardiac condition measure of the patient to the cardiac condition measure to identify a change in cardiac condition. The operations may be configured to receive, from the wearable medical device, the at least one historic cardiac condition measure. Comparing the at least one historic cardiac condition measure of the patient to the cardiac condition measure may include calculating an improvement score quantifying a difference between the at least one historic cardiac condition measure and the cardiac condition measure. The at least one historic cardiac condition measure may be a previously determined cardiac condition measure determined by applying the physiological data to the machine learning engine.

In some embodiments, the wearable medical device is a holter monitor. The wearable medical device may include a wearable cardioverter defibrillator. The wearable medical device may be configured to collect a 12 channel ECG signal. An area under the curve (AUC) performance of the machine learning engine may be at least 70%, between about 70% and about 75%, between about 75% and about 80%, or between about 80% and about 85% when classifying the approximation of the cardiac condition measure into at least two classifications. The system may include the wearable medical device.

In one aspect, the present disclosure relates to a method for remotely monitoring for improving or worsening cardiac condition in patients of a patient population fitted with wearable heart monitoring devices based on machine learning analysis, the method including for each patient of the patient population, obtaining, by processing circuitry, physiological data representing a sample timeframe, the physiological data including ECG data representing ECG signals of a patient, where the ECG signals were collected by at least two ECG electrodes of the wearable medical device monitoring a heart of the patient, and/or cardio-vibrational data representing cardio-vibrational signals of the patient, where the cardio-vibrational signals were collected by at least one vibrational sensor of the wearable medical device monitoring the heart of the patient. The method may include determining, by the processing circuitry for each patient of the patient population, an approximation of change in a cardiac condition measure of the patient, where determining includes applying the physiological data to a machine learning engine to determine the cardiac condition measure of the patient, where applying includes applying the physiological data and the cardio-vibrational data to a first tier of the machine learning engine including one or more first machine learning classifiers to obtain a first result, applying the first result along with i) clinical information regarding the patient and/or ii) physiological metrics of the patient to a second tier of the machine learning engine including one or more second machine learning classifiers to obtain a second result, and comparing at least one historic cardiac condition measure of the respective patient to the cardiac condition measure corresponding to the second result of the respective patient, where the determining results in identifying one or more patients of the patient population having worsened cardiac condition. The method may include providing, by the processing circuitry for review by a medical professional, identification of each patient of the one or more patients.

In some embodiments, the second tier includes one or more fully connected artificial neural network layers. The first tier may include one or more convolutional artificial neural network layers. In some embodiments, i) the one or more first machine learning classifiers include at least two stages of machine learning classifiers of the first tier and/or ii) the one or more second machine learning classifiers include at least two stages of machine learning classifiers of the second tier. The one or more first machine learning classifiers may include an ECG trained machine learning classifier and a cardio-vibrational data trained machine learning classifier. The one or more first machine learning classifiers may be configured to implement sets of convolutional filters, each being sized between about 2 and 10 weights, between about 10 and 50 weights, between about 50 and 100 weights, or between about 100 and 10000 weights. The one or more first machine learning classifiers may be tuned using a predetermined number of filters, where the predetermined number of filters is less than 4, between 4 and 6, between 6 and 10, or up to 15.

In some embodiments, the first result includes features extracted from the physiological data. The cardiac condition measure may be one of Class I heart failure (HF), Class II HF, Class III HF, or Class IV HF in accordance with the New York Heart Association (NYHA) classification system. The cardiac condition measure may be an ejection fraction measure ranging from 0% to 100%. The cardiac condition measure may be an ejection fraction classification. The ejection fraction classification may include one of a) an ejection fraction of greater than 30% as a first ejection fraction classification, and an ejection fraction of less than 30% as a second ejection fraction classification, b) an ejection fraction of greater than 35% as the first ejection fraction classification, and an ejection fraction of less than 35% as the second ejection fraction classification, c) an ejection fraction of greater than 40% as the first ejection fraction classification, and an ejection fraction of less than 40% as the second ejection fraction classification, or d) an ejection fraction of greater than 45% as the first ejection fraction classification, and an ejection fraction of less than 45% as the second ejection fraction classification.

In some embodiments, the method includes providing, by the processing circuitry, information regarding the cardiac condition measure to a remote computing device for review by a medical professional, clinician, or caregiver. Providing the information may include adding, for review within a medical professional patient information portal, a visual indication of worsened cardiac condition associated with each patient of the one or more patients. The method may include providing, for review within the medical professional patient information portal, a visual display representing at least a portion of the physiological metrics. The sample timeframe may be between about 10 seconds to about 20 seconds, between about 20 seconds to about 30 seconds, between about 30 seconds and about 40 seconds, between about 40 seconds and about 50 seconds, or between about 50 seconds and about 60 seconds.

In some embodiments, the physiological metrics include one or more of electromechanical activation time (EMAT), time-corrected EMAT (EMATc), left ventricular systolic time (LVST), S3 intensity, S4 intensity, S3 duration, S4 duration, systolic dysfunction index (SDI), or heart rate. The clinical information may include one or more of an age of the patient, a gender of the patient, an indication of a coronary artery bypass grafting (CABG) procedure on the patient, an indication of a congenital heart defect (CONG) diagnosis of the patient, an indication of an implantable cardioverter defibrillator explant (EXPLANT) procedure on the patient, an indication of heart failure or cardiomyopathy (HFCM) in the patient, an indication of diagnosis of a prior myocardial infarction of the patient, an indication of prior ventricular fibrillation of the patient, an indication of prior ventricular tachycardia of the patient, or an indication of diagnosis of cardiac ischemia of the patient. The ECG data and/or the cardio-vibrational data may include raw signal data collected by the wearable medical device. The method may include accessing, by the processing circuitry, the clinical information from a non-transitory computer readable storage region. The method may include receiving, from the wearable medical device of each patient of the patient population, at least a portion of the respective physiological metrics.

In some embodiments, the physiological data representing the sample timeframe is obtained from a period of at least a) one second prior to applying the physiological data to the machine learning engine, b) one hour prior to applying the physiological data to the machine learning engine, c) 24 hours to applying the physiological data to the machine learning engine, or d) 3 days prior to applying the physiological data to the machine learning engine. The ECG data may include a front-to-back ECG data signal and a side-to-side ECG signal. The one or more first machine learning classifiers may include a front-to-back ECG trained machine learning classifier and a side-to-side ECG trained machine learning classifier. The method may include calculating, by the processing circuitry from at least one of the ECG data and the cardio-vibrational data, at least a portion of the physiological metrics.

In some embodiments, the method includes receiving, from the wearable medical device, the at least one historic cardiac condition measure. The wearable medical device may be a holter monitor. The wearable medical device may include a wearable cardioverter defibrillator. The wearable medical device may be configured to collect a 12 channel ECG signal.

In some embodiments, comparing the at least one historic cardiac condition measure to the approximation of the cardiac condition measure includes calculating an improvement score quantifying a difference between the at least one historic cardiac condition measure and the approximation of the cardiac condition measure. The at least one historic cardiac condition measure may be a previously approximated cardiac condition measure. An area under the curve (AUC) performance of the machine learning engine may be at least 70%, between about 70% and about 75%, between about 75% and about 80%, or between about 80% and about 85% when classifying the approximation of the cardiac condition measure into at least two classifications.

In one aspect, the present disclosure relates to a system for remotely monitoring for improving or worsening cardiac condition in patients of a patient population fitted with wearable heart monitoring devices based on machine learning analysis, the system including a non-volatile computer readable storage medium including sets of physiological data, each set representing a respective sample timeframe of one or more sample timeframes, the physiological data including ECG data representing a ECG signals of each patient of the patient population, where the ECG signals were collected by at least two ECG electrodes of a wearable medical device monitoring a heart of the respective patient, and/or cardio-vibrational data representing cardio-vibrational signals of each patient of the patient population, where the cardio-vibrational signals were collected by at least one vibrational sensor of the wearable medical device monitoring the heart of the respective patient, and operations stored as computer executable instructions to a non-transitory computer readable media and/or encoded in hardware logic. The operations may be configured to, for each patient of the patient population, access, from the non-volatile computer readable storage medium, the physiological data of the respective patient, determine, for each patient of the patient population, an approximation of change in a cardiac condition measure of the patient, where determining includes applying the physiological data to a machine learning engine to determine the cardiac condition measure of the patient, where applying includes applying the physiological data and the cardio-vibrational data to a first tier of the machine learning engine including one or more first machine learning classifiers to obtain a first result, applying the first result along with i) clinical information regarding the patient and/or ii) physiological metrics of the patient to a second tier of the machine learning engine including one or more second machine learning classifiers to obtain a second result, and comparing at least one historic cardiac condition measure of the respective patient to the cardiac condition measure corresponding to the second result of the respective patient, where the determining results in identifying one or more patients of the patient population having worsened cardiac condition. The operations may be configured to provide, for review by a medical professional, identification of each patient of the one or more patients.

In some embodiments, the second tier includes one or more fully connected artificial neural network layers. The first tier may include one or more convolutional artificial neural network layers. In some embodiments, i) the one or more first machine learning classifiers include at least two stages of machine learning classifiers of the first tier and/or ii) the one or more second machine learning classifiers include at least two stages of machine learning classifiers of the second tier.

In some embodiments, the one or more first machine learning classifiers include an ECG trained machine learning classifier and a cardio-vibrational data trained machine learning classifier. The one or more first machine learning classifiers may be configured to implement sets of convolutional filters, each being sized between about 2 and 10 weights, between about 10 and 50 weights, between about 50 and 100 weights, or between about 100 and 10000 weights. The one or more first machine learning classifiers may be tuned using a predetermined number of filters, where the predetermined number of filters is less than 4, between 4 and 6, between 6 and 10, or up to 15. The first result may include features extracted from the physiological data.

In some embodiments, the cardiac condition measure is one of Class I heart failure (HF), Class II HF, Class III HF, or Class IV HF in accordance with the New York Heart Association (NYHA) classification system. The cardiac condition measure may be an ejection fraction measure ranging from 0% to 100%. The cardiac condition measure may be an ejection fraction classification. The ejection fraction classification may include one of a) an ejection fraction of greater than 30% as a first ejection fraction classification, and an ejection fraction of less than 30% as a second ejection fraction classification, b) an ejection fraction of greater than 35% as the first ejection fraction classification, and an ejection fraction of less than 35% as the second ejection fraction classification, c) an ejection fraction of greater than 40% as the first ejection fraction classification, and an ejection fraction of less than 40% as the second ejection fraction classification, or d) an ejection fraction of greater than 45% as the first ejection fraction classification, and an ejection fraction of less than 45% as the second ejection fraction classification.

In some embodiments, the operations are configured to provide information regarding the cardiac condition measure to a remote computing device for review by a medical professional, clinician, or caregiver. Providing the information may include adding, for review within a medical professional patient information portal, a visual indication of worsened cardiac condition associated with each patient of the one or more patients. The operations may be configured to provide, for review within the medical professional patient information portal, a visual display representing at least a portion of the physiological metrics. The sample timeframe may be between about 10 seconds to about 20 seconds, between about 20 seconds to about 30 seconds, between about 30 seconds and about 40 seconds, between about 40 seconds and about 50 seconds, or between about 50 seconds and about 60 seconds.

In some embodiments, the physiological metrics include one or more of electromechanical activation time (EMAT), time-corrected EMAT (EMATc), left ventricular systolic time (LVST), S3 intensity, S4 intensity, S3 duration, S4 duration, systolic dysfunction index (SDI), or heart rate. The clinical information may include one or more of an age of the patient, a gender of the patient, an indication of a coronary artery bypass grafting (CABG) procedure on the patient, an indication of a congenital heart defect (CONG) diagnosis of the patient, an indication of an implantable cardioverter defibrillator explant (EXPLANT) procedure on the patient, an indication of heart failure or cardiomyopathy (HFCM) in the patient, an indication of diagnosis of a prior myocardial infarction of the patient, an indication of prior ventricular fibrillation of the patient, an indication of prior ventricular tachycardia of the patient, or an indication of diagnosis of cardiac ischemia of the patient.

In some embodiments, the ECG data and/or the cardio-vibrational data includes raw signal data collected by the wearable medical device. The operations may be configured to access the clinical information from a non-transitory computer readable storage region. The operations may be configured to receive, from the respective wearable medical device of the patient, at least a portion of the physiological metrics.

In some embodiments, the physiological data representing the sample timeframe is obtained from a period of at least a) one second prior to applying the physiological data to the machine learning engine, b) one hour prior to applying the physiological data to the machine learning engine, c) 24 hours to applying the physiological data to the machine learning engine, or d) 3 days prior to applying the physiological data to the machine learning engine. The ECG data may include a front-to-back ECG data signal and a side-to-side ECG signal. The one or more first machine learning classifiers may include a front-to-back ECG trained machine learning classifier and a side-to-side ECG trained machine learning classifier.

In some embodiments, the operations are configured to calculate, from at least one of the ECG data and the cardio-vibrational data, at least a portion of the physiological metrics. The operations may be configured to receive, from the wearable medical device of the respective patient of the patient population, the at least one historic cardiac condition measure.

In some embodiments, the wearable medical device is a holter monitor. The wearable medical device may include a wearable cardioverter defibrillator. The wearable medical device may be configured to collect a 12 channel ECG signal.

In some embodiments, comparing the at least one historic cardiac condition measure to the approximation of the cardiac condition measure includes calculating an improvement score quantifying a difference between the at least one historic cardiac condition measure and the approximation of the cardiac condition measure. The at least one historic cardiac condition measure may be a previously approximated cardiac condition measure. An area under the curve (AUC) performance of the machine learning engine may be at least 70%, between about 70% and about 75%, between about 75% and about 80%, or between about 80% and about 85% when classifying the approximation of the cardiac condition measure into at least two classifications.

The foregoing general description of the illustrative implementations and the following detailed description thereof are merely exemplary aspects of the teachings of this disclosure, and are not restrictive.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute a part of the specification, illustrate one or more embodiments and, together with the description, explain these embodiments. The accompanying drawings have not necessarily been drawn to scale. Any values dimensions illustrated in the accompanying graphs and figures are for illustration purposes only and may or may not represent actual or preferred values or dimensions. Where applicable, some or all features may not be illustrated to assist in the description of underlying features. In the drawings:

FIG. 1A and FIG. 1B illustrate flow diagrams of example processes for determining a cardiac condition measure of a patient through applying a multi-tier machine learning scheme to data collected by a wearable medical device donned by the patient;

FIG. 2A and FIG. 2B illustrate waveforms representing example raw data collected by a wearable medical device donned by a patient;

FIG. 3 is a flow diagram of an example process for calculating a cardiac condition measure of a patient using multiple rounds of machine learning analysis;

FIG. 4 is a flow diagram of an example process for monitoring a patient population for evidence of a worsening cardiac condition measure;

FIG. 5 illustrates an example process for developing training data for a multi-tier machine learning scheme for determining a cardiac condition measure of a patient;

FIG. 6A and FIG. 6B present summary information of example data sets used for training and testing embodiments of multi-tier machine learning schemes;

FIG. 7 presents summary information of test results for embodiments of multi-tier machine learning schemes;

FIG. 8A and FIG. 8B illustrate example cardiac condition measure calculations over time for a first and second patient population;

FIG. 8C illustrates a graphical distribution of ejection fraction determinations including determinations made using data from a beginning of use period of time in comparison to data from an end of use period of time;

FIG. 9 illustrates a graphical distribution of ejection fraction values in comparison to determined cardiac condition measures;

FIG. 10 is a block diagram of an example medical device for monitoring a cardiac condition of a patient; and

FIGS. 11A through 11D illustrate example wearable medical devices for monitoring a cardiac condition of a patient.

### DETAILED DESCRIPTION OF ILLUSTRATIVE EMBODIMENTS

The description set forth below in connection with the appended drawings is intended to be a description of various, illustrative embodiments of the disclosed subject matter. Specific features and functionalities are described in connection with each illustrative embodiment; however, it will be apparent to those skilled in the art that the disclosed embodiments may be practiced without each of those specific features and functionalities.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments. Further, it is intended that embodiments of the disclosed subject matter cover modifications and variations thereof.

It must be noted that, as used in the specification and the appended claims, the singular forms “a,” “an,” and “the” include plural referents unless the context expressly dictates otherwise. That is, unless expressly specified otherwise, as used herein the words “a,” “an,” “the,” and the like carry the meaning of “one or more.” Additionally, it is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Furthermore, the terms “approximately,” “about,” “proximate,” “minor variation,” and similar terms generally refer to ranges that include the identified value within a margin of 20%, 10% or preferably 5% in certain embodiments, and any values therebetween.

In one aspect, the present disclosure relates to methods, systems, and a platform environment for monitoring a patient or group of patients in a patient population for signs of change in cardiac condition without requiring the patient(s) to journey to a medical facility. The methods and systems, for example, may replace and/or supplement periodic echocardiogram tests or basic metabolic panel (BMP) tests that may otherwise be recommended to derive present status of a patient's cardiac condition, saving money for both patients and medical facilities and avoiding relying on patient compliance with these periodic visits. Further, the methods and systems described herein may provide earlier identification of worsening of a heart condition than otherwise enabled through periodic medical facility visits.

In some embodiments, sensor data obtained from sensors of a wearable medical device monitoring the heart of patient is used to supply information for analysis by artificial intelligence. The wearable medical device, in some examples, may be a Holter monitor, a wearable cardioverter defibrillator, or other wearable cardiac monitor, examples of which are provided in greater detail below. The information is provided to a machine learning engine trained to determine one or more cardiac condition measures indicative of, in some examples, heart failure classification or ejection fraction (EF). Through monitoring the cardiac condition measure(s), medical personnel, for example, can identify those patients more likely to require an intervention, office visit, or other medical procedure. In illustration, the monitoring may be used to identify candidates for implantable medical devices due to worsening of a heart condition. Further, the monitoring may be used to track likelihood of compliance with a prescribed routine for supporting heart health and/or slowing advancement of heart failure. For example, through monitoring the cardiac condition measure(s) remotely, medical personnel may issue reminders or other intervention to keep patients on track with exercises, lifestyle changes, medications, and/or supplements that promote stabilization or improvement of the heart condition.

In some embodiments, an artificial intelligence engine including at least two tiers of machine learning classifier application is used to approximate a present measure or category of measure of a cardiac condition without access to data traditionally applied to derive the cardiac condition measure. The artificial intelligence engine, for example, may be designed to analyze ECG samples and/or cardio-vibrational samples obtained by a wearable medical device monitoring the patient's heart in a first tier of analysis that applies a first classifier or set of classifiers and produces a first result. The first result may be provided to a second tier of analysis, along with physiological metrics and/or clinical information regarding the patient, to be applied to a second classifier or set of classifiers used to produce the cardiac condition measure. In this manner, the approximation of the current cardiac condition measure is determined, albeit with somewhat lower accuracy than a traditional laboratory or clinical analysis, but while the patient is allowed to go about their typical activities when wearing the wearable medical device.

In one aspect, the present disclosure relates to methods, systems, and a platform environment for performing periodic, ongoing monitoring of a patient or group of patients fitted with wearable cardiac monitoring devices while going about daily activities in and around the home environment. The ongoing monitoring, in some examples, may be performed daily, at multiple times per day, hourly, or multiple times per hour. This differs from traditional monitoring of the cardiac condition measure which is otherwise achievable only in a clinical or laboratory setting. Through ongoing monitoring of the patient, changes in the approximation of the cardiac condition measure can provide clinicians and/or other medical professionals with a strong understanding of changes in cardiac condition in the patient despite some reduction in accuracy of individual determinations of the cardiac condition measure in comparison to the traditional calculation. Further, the availability of additional physiological metrics calculated by the wearable medical device may be used not only to enhance the analysis provided by the artificial intelligence engine as applied to the second tier of machine learning analysis but also to enhance a clinician's or other professional's interpretation of the cardiac condition measure and the trends in measurements when provided in a patient portal or other report summary. In illustration, based upon a present cardiac condition measure or trend in cardiac condition measure, a patient may be identified to the clinician or medical professional, for example in a report or patient portal interface, as requiring closer attention or intervention. In this manner, a clinician or medical professional with a busy practice may easily focus on one-on-one visits or other intervention with those patients in greatest need of attention, rather than needing to schedule patient visits for monitoring purposes to collect information regarding the cardiac condition measure.

Conventionally, for ECG monitoring skin-facing ECG electrodes of a medical device are positioned at the following standard anatomical locations: Right Arm (RA), Right Leg (RL), Left Arm (LA), Left Leg (LL), fourth intercostal space on the right sternum (V₁), fourth intercostal space at the left sternum (V₂), midway between placement of V₂ and V₄ (V₃), fifth intercostal space at the midclavicular line (V₄), anterior axillary line on a same horizontal level as V₄ (V₅), and at mid-axillary line on a same horizontal level as V₄ and V₅ (V₆). These anatomical locations are described in Table 1 below.

TABLE 1

| Electrode name | Description of standard anatomical location |
|---|---|
| RA | On the right arm, avoiding thick muscle. |
| LA | In the same location where RA was placed, |
| | but on the left arm. |
| RL | On the right leg, lower end of medial aspect |
| | of calf muscle. (Avoid bony prominences) |
| LL | In the same location where RL was placed, |
| | but on the left leg. |
| V1 | In the fourth intercostal space (between ribs 4 and 5) |
| | just to the right of the sternum (breastbone). |
| V2 | In the fourth intercostal space (between ribs 4 and 5) |
| | just to the left of the sternum. |
| V3 | Between leads V2 and V4. |
| V4 | In the fifth intercostal space (between ribs 5 and 6) |
| | in the mid-clavicular line. |
| V5 | Horizontally even with V4, in the left |
| | anterior axillary line. |
| V6 | Horizontally even with V4 and V5 in |
| | the midaxillary line. |

Other standard ECG lead systems include a 3 lead ECG system and a 5 lead ECG system. For example, the 3 lead ECG system can include 3 or 4 ECG electrodes. These electrodes are located as follows: Right Arm (RA), Left Arm (LA), Left Leg (LL), and Right Leg (RL). These leads can provide information for basic heart rhythm monitoring but not for certain specialized features. For example, a specialized feature such as ST segment elevation may not be detected by a 3 lead ECG system. This is because the three leads do not provide adequate information about the anterior wall of the heart. For example, the 5 lead ECG system can use 4 extremity leads and one precordial lead. In implementations, the 5 electrodes include Right Arm (RA), Left Arm (LA), Right Leg (RL), Left Leg (LL), and one precordial chest electrode (V₁) although, in certain standard 5 ECG lead systems, the 5^{th} lead electrode can be changed.

However, to monitor patients over days, weeks, months, or even years, in some embodiments, the wearable heart monitoring medical device may be designed for comfort and durability, differing from traditional ECG monitoring by establishing fewer electrical channels (leads) and/or establishing the electrical channels through non-conventional electrode placement (e.g., positioned in accordance with one or more non-standard ECG leads). For example, non-standard leads include one or more leads that do not correspond to any of the 12 standard ECG leads listed in Table 1 above. In some embodiments, the non-standard leads when represented as vectors in a vectorcardiogram are within 15° of any of the standard ECG leads. The skin-facing electrodes of the wearable medical device may be located at anatomical sites on the left or right side of the patient's torso. In such locations, the skin-facing electrodes form one or more non-standard (e.g., side-to-side (SS) and/or front-to-back (FB)) ECG leads for monitoring the electrical activity of the heart. Having non-standard placement of electrodes as described above, the medical device, in some implementations, is configured to identify one or more non-standard ECG vectors and to determine, based on sensed ECG data, a first one or more ECG leads for continuous monitoring of the patient's heart activity. The ECG leads can be represented as vectors in a vectorcardiogram representation of the electrical activity of the heart.

As implemented for testing purposes, the results of which are described in detail below, ECG and/or cardio-vibrational samples obtained through monitoring a patient population that have worn the LifeVest® wearable cardioverter defibrillator (WCD) device over a certain span of years (e.g., patients from between 2014 and 2020) were used to train machine learning classifiers of the artificial intelligence engine. The LifeVest® WCD, for example, includes both a side-to-side non-conventional ECG lead and a front-to-back (FB) non-conventional ECG lead. As evidenced by the test results presented below, the data collected through daily wear monitoring of LifeVest® WCD patients, when applied to the machine learning engine as described within this disclosure, supplies adequate information to determine ejection fraction measurements with clinically applicable precision while allowing the patients the freedom and convenience of being monitored within the home environment without clinical assistance in lead positioning.

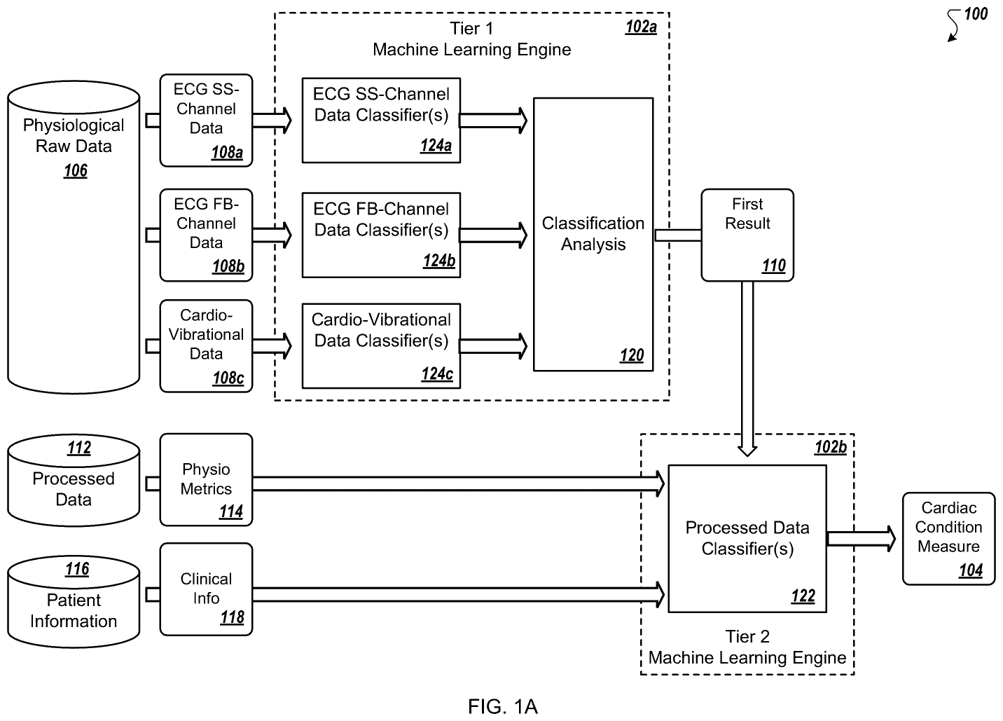

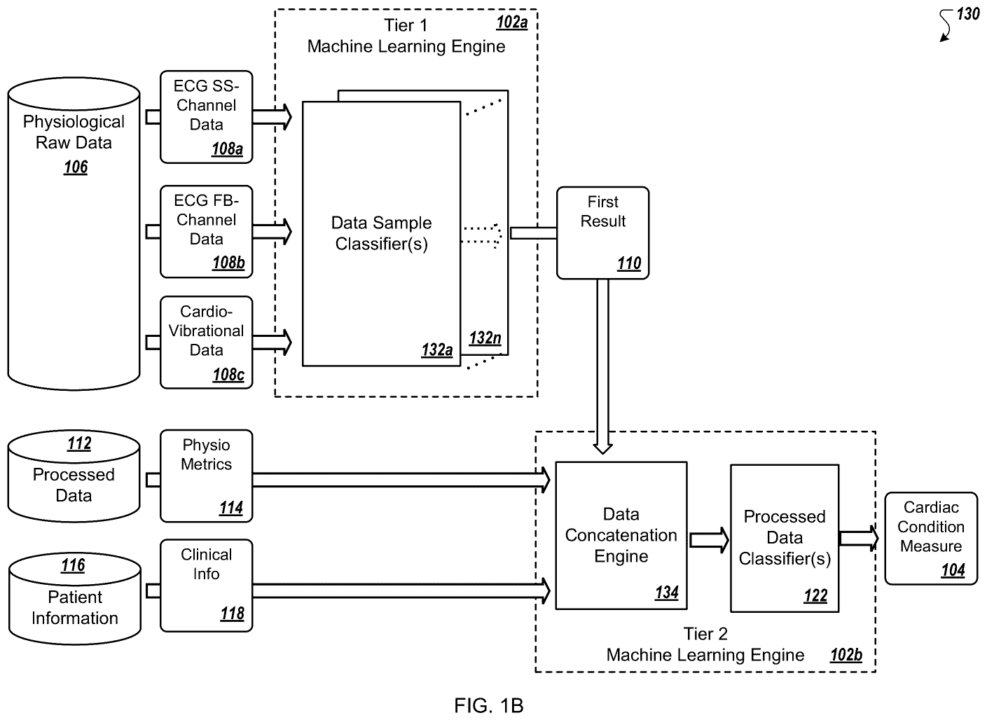

FIG. 1A and FIG. 1B illustrate flow diagrams of example processes 100 and 130 for determining a cardiac condition measure 104 of a patient through applying a multi-tier machine learning engine 102 to physiological data 106 collected by a wearable medical device donned by the patient. In the multi-tier machine learning engine 102, in some embodiments, data samples 108 derived from raw physiological data 106 captured by a wearable medical device, such as a first (e.g., side-to-side) ECG channel data sample 108a, a second (e.g., front-to-back) ECG channel data sample 108b, and/or a cardio-vibrational data sample 108c, are applied to a first tier 102a of the machine learning engine 102 to obtain a first result 110. The first result 110 may then be applied to a second tier 102b of the machine learning engine 102, along with patient physiological metrics 114 and/or patient clinical information 118 to determine the cardiac condition measure 104.

The cardiac condition measure, in some embodiments, is an ejection fraction (EF) measure. EF, representing the efficiency of blood ejection from a filled heart ventricle during a heartbeat, is typically measured using imaging techniques, such as echocardiogram, computerized tomography (CT) scan, cardiac catheterization, or nuclear medicine scan. Each of these measurements involves a clinical procedure. The ejection fraction measure, in some examples, may be an ejection fracture percentage ranging from 0% to 100% or an ejection fracture classification identifying the EF measure as belonging in one of two or more ranges of EF measures. The ranges, in some example, may include one of the following: an ejection fraction of greater than 30% as a first ejection fraction classification and an ejection fraction of less than 30% as a second ejection fraction classification; an ejection fraction of greater than 35% as the first ejection fraction classification and an ejection fraction of less than 35% as the second ejection fraction classification; an ejection fraction of greater than 40% as the first ejection fraction classification and an ejection fraction of less than 40% as the second ejection fraction classification; or an ejection fraction of greater than 45% as the first ejection fraction classification and an ejection fraction of less than 45% as the second ejection fraction classification.

In other embodiments, the cardiac condition measure is a heart failure classification measure. The heart failure classification measure, for example, may be one of Class I heart failure (HF), Class II HF, Class III HF, or Class IV HF in accordance with the New York Heart Association (NYHA) classification system. In another example, the heart failure classification measure may be one of Stage A HF, Stage B HF, Stage C HF, or Stage D HF according to the American College of Cardiology/American Heart Association classification system. Heart failure classification, traditionally, is gauged on a number of metrics and/or clinical observations such as patient symptoms. The metrics may include, in some examples, EF, blood pressure, oxygen consumption (VO₂), blood biomarkers, pulmonary artery pressure, transthoracic impedance, lung fluid content, heart rate, and/or metrics derived from electrocardiogram. Symptoms may include shortness of breath, fatigue, recent weight gain, abdominal swelling, edema, and/or chest tightness.

Rather than requiring clinical observation and/or testing, in some embodiments, the processes 100 and 130 rely on artificial intelligence to derive signal trends indicative of one or more of the above example cardiac condition measures. The data samples 108 used in this analysis may each represent a same sample timeframe as captured by the wearable medical device. The sample timeframe, in some examples, is between about 10 seconds to about 20 seconds, between about 20 seconds to about 30 seconds, between about 30 seconds and about 40 seconds, between about 40 seconds and about 50 seconds, or between about 50 seconds and about 60 seconds. The data samples 108 may have been captured, in some examples, within seconds of applying the physiological data to the machine learning engine (e.g., in “real-time” or “near real-time”), within one hour of applying the physiological data to the machine learning engine, within 24 hours of applying the physiological data to the machine learning engine, or between 1 and 3 days prior to applying the physiological data to the machine learning engine. The capture lag, for example, may be representative of common transfer periods for transferring data from the wearable medical device to a remote computing for analysis. In some implementations, the wearable medical device has an open data communication path with the remote system via a network, allowing for real-time or near real-time analysis. The wearable medical device, in other implementations, may periodically upload (e.g., conduct a bulk transfer) of data to a remote computing system via a network connection. While described as a network connection between the wearable medical device and the remote computing system, in some embodiments, a second computing device may be involved in the data upload to the remote computing system. For example, a smart phone or other computing device may establish a local connection (e.g., using a Wi-Fi connection, Bluetooth connection, or other short-range wireless connection) with the wearable medical device to enable transfer of the data between the wearable medical device and the remote computing system. In further implementations, at least a portion of the analysis may be performed by processing circuitry of the wearable medical device and/or another portable computing device in wired or wireless communication with the wearable medical device. Network communication examples are described in further detail below.

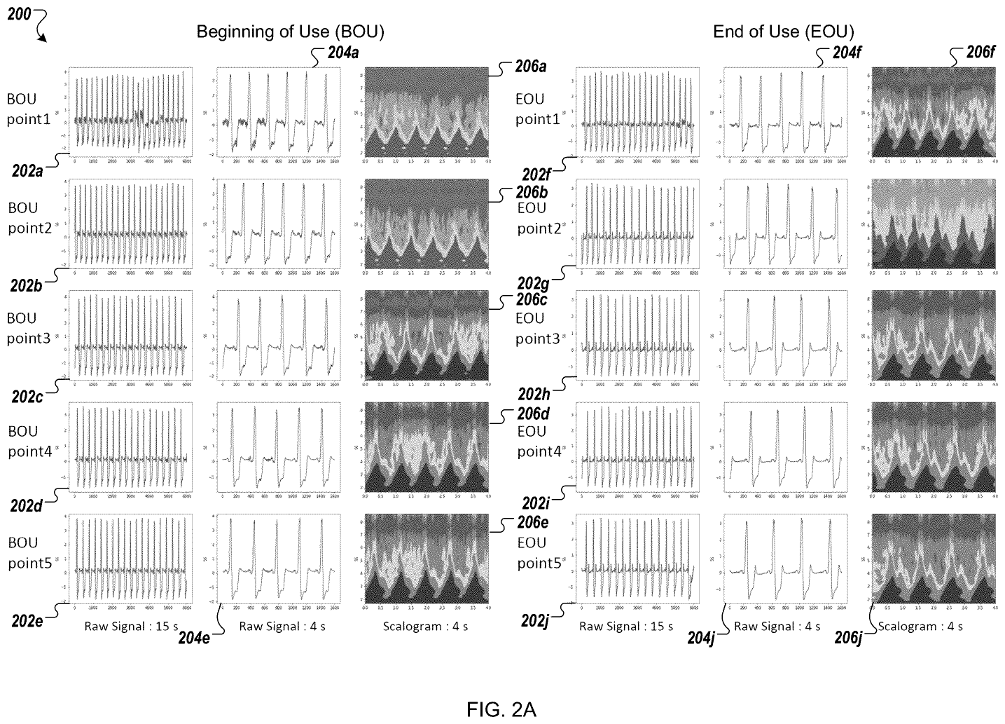

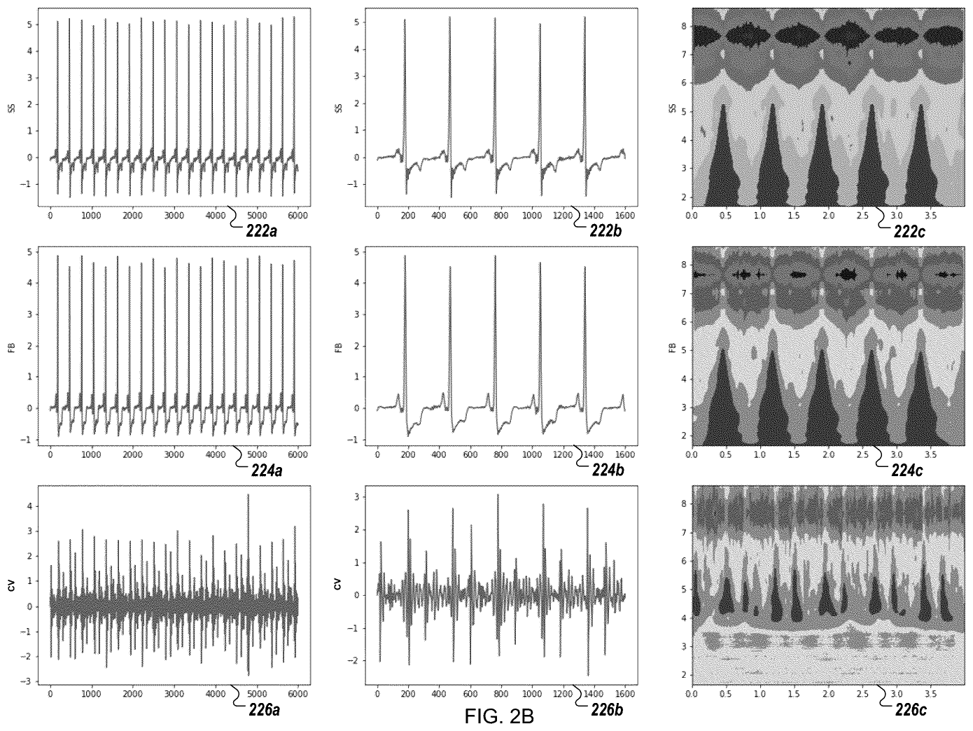

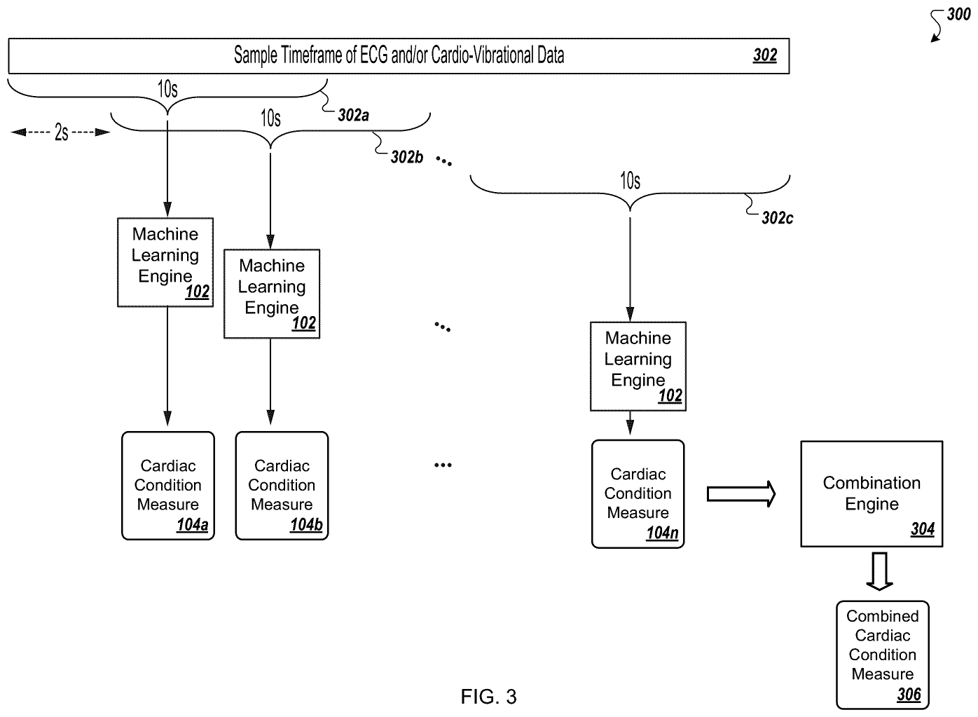

Turning to FIG. 2A, example training data 200 illustrates first ECG (e.g., 15-second data) samples 202a through 202j, second ECG (e.g., 4-second data) samples 204a through 204j, and scalogram samples 206a through 206j. Each data sample may represent a different discrete timeframe of capture. In another example, overlapping timeframes of data samples may be provided, such as overlapping data samples 302 illustrated in FIG. 3. The first and second ECG data samples 202, 204 of FIG. 2A, for example, may represent two separate ECG channels, such as a front-to-back ECG channel and a side-to-side ECG channel, similar to the ECG side-to-side sample data 108a and the ECG front-to-back sample data 108b of FIG. 1A and FIG. 1B. A data set including the first and second ECG data samples 202, 204, for example, may be provided to train both a first (e.g., side-to-side channel) data classifier and a second (e.g., front-to-back channel) data classifier of a fully connected machine learning model. The scalogram samples 206a through 206j illustrate a transformation of raw ECG signals into a frequency domain representation. A data set including the scalogram samples 206, for example, may be provided to train a third (e.g., frequency domain) ECG data classifier of a fully connected machine learning model. The scalogram samples 206, for example, may be produced from physiological raw data 106 collected by the wearable medical device prior to analysis by the multi-tier machine learning engine 102. The scalogram samples 206, for example, may each represent a wavelet transformation output at multiple times and multiple time scales. ECG data sample 202 shows one such time scale and ECG data sample 204 shows the same ECG at a finer time scale. Other transformations may be applied to the ECG side-to-side sample data 108a and/or the ECG front-to-back sample data 108b of FIG. 1A and FIG. 1B, such as transformations to reduce noise/artifacts and/or to reduce size and complexity of data being transferred across a network for analysis. The transformations, in some embodiments, match transformations applied to input data samples in training the various classifiers 124a, 124b, and 124c to ensure a consistent recognition during analysis of incoming samples.

Turning to FIG. 2B, an example set of input data includes a side-to-side ECG data sample set 222, a front-to-back ECG data sample set 224, and a cardio-vibrational data sample set 226. Each data sample set 222, 224, and 226 includes the following: a 15 second data sample 222a, 224a, 226a; a 4 second data sample 222b, 224b, 226b; and a scalogram sample 222c, 224c, 226c. Thus, the data sample sets 222, 224, 226 of FIG. 2B illustrate that each sample type may be independently transformed into a scalogram format. Further, the cardio-vibrational data sample 108c of FIG. 1A, may include, in some embodiments, one or more of the cardio-vibrational data sample types illustrated in the cardio-vibrational data sample set 226 of FIG. 2B. In training a machine learning model, for example, nine separate classifiers (e.g., nine stages of a fully connected machine learning model) may be produced corresponding to each type of sample of each data set. Thus, further to the example, the resulting fully connected machine learning model may include a coarse time scale side-to-side ECG data classifier corresponding to data type 222a, a fine time scale side-to-side ECG data classifier corresponding to data type 222b, a frequency domain side-to-side ECG data classifier corresponding to data type 222c, a coarse time scale front-to-back ECG data classifier corresponding to data type 224a, a fine time scale front-to-back ECG data classifier corresponding to data type 224b, a frequency domain front-to-back ECG data classifier corresponding to data type 224c, a coarse time scale cardio-vibrational data classifier corresponding to data type 226a, a fine time scale cardio-vibrational data classifier corresponding to data type 226b, and a frequency domain cardio-vibrational data classifier corresponding to data type 226c.

As illustrated in each of FIG. 1A and FIG. 1B, the first tier 102a of the machine learning engine 102 includes machine learning classifiers (124 or 132) for analyzing the data samples 108. Turning to FIG. 1A, in some embodiments, the first tier 102a of the machine learning engine 102 includes separate machine learning classifiers 124 for each type of data sample 108 provided to the first tier 102a. As illustrated, the first tier 102a includes at least one ECG SS-channel data classifier 124a configured to receive and analyze the ECG SS-channel data sample(s) 108a, at least one ECG FB-channel data classifier 124b configured to receive and analyze the ECG FB-channel data sample(s) 108b, and at least one cardio-vibrational data classifier 124c configured to receive and analyze the cardio-vibrational data sample(s) 108c. The individual classifiers 124 may be executed in parallel or in serial, depending on implementation. In other embodiments, turning to FIG. 1B, the first tier 102a includes one or more machine learning classifiers 132a through 132n each configured to accept one or more types of data samples 108. For example, the data sample classifiers 132 may be developed to filter combined data samples 108a serially.

The classifiers 124 of FIG. 1A and/or 132 of FIG. 1B, in some embodiments, each represent one or more convolutional neural network (CNN) stages of a CNN model. CNN models, for example, are designed to break down features of graphical or image data, such as the time domain ECG data sample graphs 222a and 222b and/or the frequency domain scalogram 222c of the side-to-side data samples 222 of FIG. 2B, into sub-features. This makes CNN classification particularly advantageous for image classification. Further, CNN models, as opposed to fully connected artificial neural network layers, can be used to constrain the output space, thereby reducing the complexity and processing demands. This can be advantageous with large data sets such as raw ECG and cardio-vibrational signals. In some implementations, the CNN model may include a network in network (NiN) model. NIN models, for example, take CNN processing to another level by analyzing a network of convolutional layers of the input data. The particular architecture of CNN model being applied, in some examples, may be based in part on processing availability and/or storage size availability in the end system (e.g., a cloud network versus processing on a medical device), third party tool access (e.g., availability of cloud provider specialized tools and hardware for performing image classification), and/or processor type (e.g., GPU, CPU, FPGA, etc.). Further, the architecture of the CNN model may be selected in part based upon the complexity and truth data set required to train the model. CNN models, as opposed to fully connected artificial neural network layers, for example, are typically easier to train and can generate good results based on more limited truth data sets.

The classifiers 124 of FIG. 1A and/or 132 of FIG. 1B, in some implementations, implement sets of convolutional filters. Each filter, for example, may be sized between about 2 and 10 weights, between about 10 and 50 weights, between about 50 and 100 weights, or between about 100 and 10000 weights. In some embodiments, the classifiers 124 of FIG. 1A and/or 132 of FIG. 1B are tuned using a predetermined number of filters, wherein the predetermined number of filters is less than 4, between 4 and 6, between 6 and 10, or up to 15.

As illustrated in FIG. 1A, in some embodiments, the output of each of the machine learning classifiers 124a, 124b, and 124c is provided to a classification analysis engine 120 to produce a first result 110. The classification analysis engine 120, for example, may represent an additional one or more CNN stages of the CNN model, designed to combine the output of the classifiers 124 into an output representative of one or more features most relevant to the determination of the cardiac measure. Conversely, as illustrated in FIG. 1B, in other embodiments, the output of the machine learning classifiers 132 may directly produce the first result 110. The first result 110, in some examples, may be a feature vector, or feature map (e.g., matrix) including features extracted from the physiological data 106 (e.g., features of the ECG SS-channel data sample 108a, the ECG FB-channel data sample 108b, and/or the cardio-vibrational data sample 108c). The feature vector or feature matrix, for example, may include ECG and/or cardio-vibrational features most relevant to the determination of the cardiac measure.

As illustrated in both FIG. 1A and FIG. 1B, in some embodiments, the first result 110 is provided with physiological metrics 114 and/or clinical information 118 to the second tier 102b of the machine learning engine 102. In some examples, the physiological metrics 114 may include cardiac acoustical biomarkers such as electromechanical activation time (EMAT), time-corrected EMAT (EMATc), left ventricular systolic time (LVST), S3 intensity, S4 intensity, S3 duration, S4 duration, and/or systolic dysfunction index (SDI). The physiological metrics 114 may include, in further examples, heart rate (HR), blood pressure (BP), respiratory rate, and/or body temperature. A portion of the physiological metrics 114, in some embodiments, are derived from the raw physiological data 106 captured by the wearable medical device (e.g., as processed data 112). Further, a portion of the physiological metrics 114 may be accessed from additional sensor data collected by the wearable medical device or another device monitoring the patient. The clinical information 118 may include demographic metrics such as age of the patient and/or gender of the patient. Further, the clinical information 118 may include diagnoses applied to the patient such as, in some examples, an indication of a congenital heart defect (CONG) diagnosis of the patient, an indication of heart failure or cardiomyopathy (HFCM) in the patient, an indication of diagnosis of a prior myocardial infarction of the patient, an indication of prior ventricular tachycardia of the patient, or an indication of diagnosis of cardiac ischemia of the patient. Additionally, the clinical information 118 may include past medical procedures of the patient such as, in some examples, an indication of prior ventricular fibrillation of the patient, an indication of a coronary artery bypass grafting (CABG) procedure on the patient, and/or an indication of an implantable cardioverter defibrillator explant (EXPLANT) procedure on the patient. The clinical information 118 may be accessed from patient information 116 (e.g., medical records) stored separately from the wearable medical device and/or from data retained by the wearable medical device itself.

Turning to FIG. 1B, in some implementations, the second tier 102b of the machine learning engine 102 includes a data concatenation engine 134 configured to concatenate the first result 110, the physiological metrics 114, and/or the clinical information 118 into a feature vector or feature map (e.g., matrix) form for analysis by one or more processed data classifiers 122. In some examples, the data concatenation engine 134 may be similarly included in the architecture of FIG. 1A. The data concatenation engine 134, in some embodiments, merges the first result 110 with the physiological metrics 114 and/or clinical information 118 by conforming inputs to a format anticipated by the processed data classifier(s) 122. For example, the indications of diagnoses and/or prior medical procedures may be supplied to the data concatenation engine 134 as and/or converted by the data concatenation engine 134 into a binary feature vector of flags (e.g., yes/no). In another example, the patient's age may be converted into an age classification of two or more age ranges. Other data manipulations are possible.

As illustrated in both FIG. 1A and FIG. 1B, in some embodiments, the processed data classifier(s) 122 analyze the combined information of the first result 110 with the physiological metrics 114 and/or clinical information 118 to determine the cardiac condition measure 104. The processed data classifier(s) 122, for example, may represent one or more fully connected (FC) neural network stages of a convolutional model. Because the input data (e.g., physiological metrics 114 and/or clinical information 118) provided to the second tier 102b of the machine learning engine 102 is simpler and less diverse, for example, the FC stages of the convolutional neural network model can be trained and applied without requiring a large amount of time or resources. Further, the refined analysis provided by the FC neural network stage(s) may increase accuracy of the determination of the cardiac condition measure 104.

Although described in relation to a two-tier machine learning engine, in further embodiments, the machine learning engine 102 may include three or more tiers. For example, the ECG related data samples 108a, 108b may be analyzed by a separate machine learning tier than the cardio-vibrational data samples 108c. Further, the physiological metrics 114 (in combination with the first result 110) may be analyzed by a separate machine learning tier than the clinical information 118 (in combination with the first result 110). Other modifications are possible.

Although the process flows 100 and 130, respectively, have been described as analyzing a particular set of data samples acquired at a particular timeframe to determine the cardiac condition measure 104, in some embodiments, signal data over a predetermined time period may be divided into multiple data samples, with each data sample being applied to the machine learning engine 102 to obtain a set of cardiac condition measures 104 that may then be combined to determine a final cardiac condition measure. In this way, for example, transient anomalies in collected data may be thrown out or smoothed over in determining the cardiac condition measure.

Turning to FIG. 3, an example process flow 300 is presented for calculating a cardiac condition measure of a patient by using multiple rounds of machine learning analysis. A sample timeframe of physiological data, including ECG data and/or cardio-vibrational data, in some implementations, is separated into data sample 302a through data sample 302n, each data sample 302 representing a same sample length of time (e.g., ten seconds). The data samples 302a through 302n may represent, for example, the ECG SS-channel data sample 108a, the ECG FB data sample 108b, and/or the cardio-vibrational data sample 108c as described in relation to FIG. 1A. The data samples 302a through 302n may be divided to form samples having overlapping time periods. As illustrated, for example, a beginning of the second data sample 302b is offset in time by two seconds from the first data sample 302b, such that data sample 302a and data sample 302b share a same six seconds of data signals.

Each of the data samples 302a through 302n, in some implementations, is provided to the machine learning engine 102 (e.g., according to the process flow 100 of FIG. 1A or the process flow 130 of FIG. 1B) to generate respective cardiac condition measures 104a through 104n. Calculation of the cardiac condition measures 104a through 104n may be performed serially or in parallel, for example depending upon data acquisition. In an illustrative embodiment, as the sample timeframe of physiological data 302 is acquired, real-time or near real-time analysis may be performed by the process flow 300 through launching artificial intelligence analysis by the machine learning engine 102 on each portion 302a through 302n of the sample timeframe of physiological data 302 as the portion is obtained from the incoming sample timeframe of physiological data 302. In some embodiments, the same physiological metrics (e.g., physiological metrics 114 of FIG. 1A and FIG. 1B) and/or the same clinical information (e.g., clinical information 118 of FIG. 1A and FIG. 1B) may be applied during each round of machine learning analysis while analyzing the extent of the sample timeframe of physiological data 302.

In some implementations, a combination engine 304 combines the cardiac condition measures 104a through 104n to determine a combined cardiac condition measure 306. The combination engine 304, in some examples, may mathematically combine the cardiac condition measures 104a through 104n to determine an average or median value. Further, the combination engine 304 may discard one or more data points of the cardiac condition measures 104a through 104n as being outlier measures exceeding a range of anticipated values. For example, if a certain one of the cardiac condition measures 104a through 104n is less than and/or greater than a threshold distance from a median value of the cardiac condition measures 104a through 104n, that cardiac condition measure may be removed from consideration in determining the combined cardiac condition measures 306. In other examples, any cardiac condition measure 104a through 104n outside of a predetermined range of values or exceeding a threshold range from a previously determined combined cardiac condition measure 306 (e.g., from an hour prior, a day prior, etc.) may be discarded as outlying data.

The cardiac condition measure, in some implementations, is applied in determining health-related prompts to provide to the patient. For example, the wearable medical device or another patient device, such as a smart phone application in communication with the wearable medical device, may include prompts for directing the patient to perform certain activities, such as walking exercises and/or breathing exercises. In another example, the wearable medical device or other patient device may prompt the patient and/or the patient's caregiver to perform a health status check (e.g., blood pressure monitoring, oxygen level monitoring, etc.). Further, the cardiac condition measure may be used to prompt scheduling of a doctor visit.

In some implementations, the cardiac condition measure is shared with one or more clinicians or other medical professionals, such as the patient's primary care doctor or cardiologist. The cardiac condition measure, for example, may be calculated by an external computing system and/or uploaded to an external computing system for use in remote monitoring of the patient. Other data and metrics, such as the physiological data, physiological metrics, and/or the physiological data samples (e.g., ECG and/or cardio-vibrational data) may be provided to an external computing system as well for remote monitoring. The cardiac condition measure, for example, may be used to inform a clinician or medical professional regarding worsening of the patient's condition, allowing the clinician or medical professional to provide timely intervention to the patient.

In some implementations, rather than or in addition to sharing the cardiac condition measure, a difference between the present cardiac condition measure and one or more previously calculated (e.g., historic) cardiac condition measures is calculated. The difference, for example, may be considered as a percentage change. In another example, the difference may be quantified as a score (e.g., an improvement score) identifying a worsening or improving cardiac condition. The historic cardiac condition measures may include cardiac condition measures approximated using a machine learning process as described herein and/or cardiac condition measures directly measured using equipment or tests in a clinical setting.

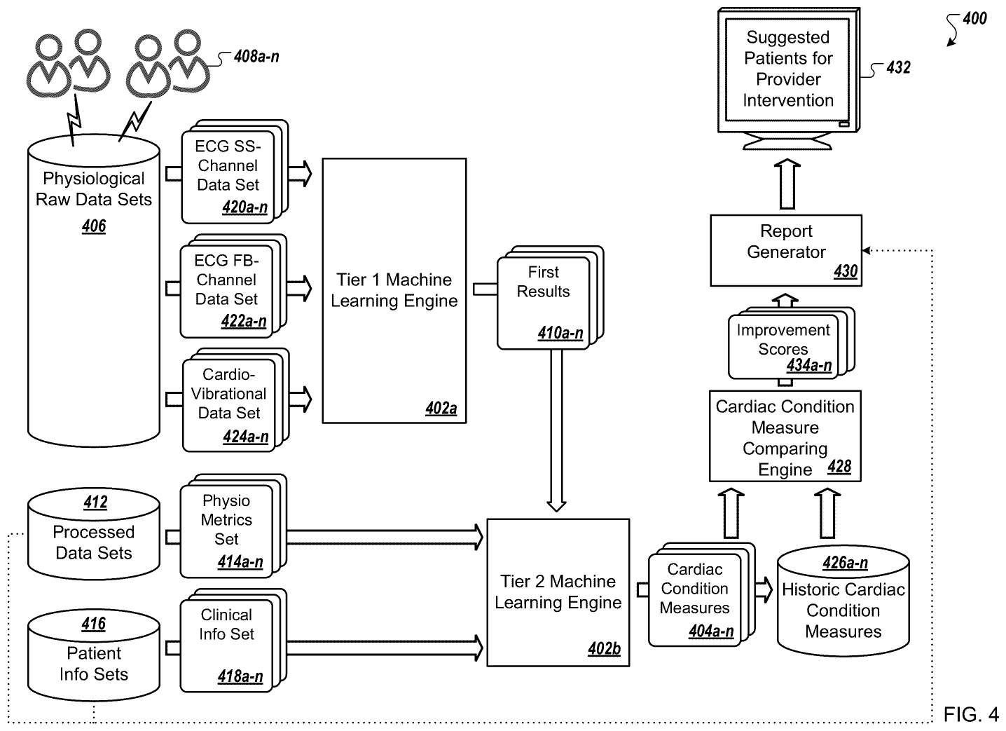

FIG. 4 illustrates an example process 400 for monitoring a patient population 408a-n for evidence of a worsening cardiac condition measure. The example process 400 of FIG. 4 relies on a multi-tier machine learning engine 402a and 402b for analyzing data samples (e.g., ECG SS-channel data samples 420a-n, ECG FB-channel samples 433a-n, cardio-vibrational data samples 424a-n) obtained from the patient population 408a-n along with physiological metrics 414a-n and patient information 416a-n to determine cardiac condition measures 404a-n corresponding to each member of the patient population 408a-n, as described above in relation to FIG. 1A through FIG. 3. Further, according to the example process 400, the cardiac condition measures 404a-n are analyzed in view of historic cardiac condition measures 426a-n to generate improvement scores 434a-n which can be applied to a presentation to a clinician or caregiver for tracking patient population cardiac conditions and identifying those patients of the patient population 408a-n in need of medical attention or other interventions. The process 400 can provide the benefit of identifying, to a physician or other medical professional with a busy practice, those patients in need of an in-person visit, rather than placing all cardiac patients on a same visit schedule. This can free medical professionals to take on additional patients while ensuring those patients in greatest need are promptly treated. Further, the process 400 may assist in prompting physician feedback. For example, upon evidence of a worsening cardiac condition (or unchanged), the physician may prompt the patient to perform additional rehabilitation activities, such as six-minute walk tests or other exercises. Conversely, upon evidence of an improving cardiac condition, the physician may be prompted to provide positive feedback to the patient for the patient's rehabilitation efforts.

Turning to FIG. 4, after the determination of the cardiac condition measures 404a-n by the second tier 402b of the machine learning engine 402, in some implementations, the cardiac condition measures 404a-n are provided to a cardiac condition measure comparing engine 428 to compare each of the cardiac condition measures 404a-n to one or more historic cardiac condition measures 426a-n of the patient. The historic cardiac condition measures 426a-n may include cardiac condition measures previously determined by the machine learning engine 402. For example, the cardiac condition measures 404a-n may be stored as a most recent copy of historic cardiac condition measures 426a-n for future use. In another example, the historic cardiac condition measures 426a-n may include cardiac condition measures directly measured using equipment or tests in a clinical setting. The cardiac condition measure comparing engine 428 analyzes, for each patient of the patient population 408a-n, the most recently determined cardiac condition measures 404a-n in view of one or more historic cardiac condition measures 426a-n corresponding to the respective patient to determine a respective improvement score 434a-n of the respective patient. The cardiac condition measure comparing engine 428, in some examples, may determine an absolute value of change, a rate of change, and/or a percentage change in comparison to the one or more historic cardiac condition measures 426a-n. In another example, the cardiac condition measure comparing engine 428 may determine a current score within a range of scores as well as a difference in score between a prior score and a current score.

In some implementations, the improvement scores 434a-n are provided to a report generator 430 to generate a message, report, and/or interactive graphical user interface 432 for review by a medical professional or clinician. The report generator 430, for example, may flag for identification one or more patients of the patient population 408a-n having a worsened cardiac condition according to the improvement scores 434a-n. The report generator 430 may further present physiological data (e.g., ECG SS-channel data samples 420a-n, ECG FB-channel samples 433a-n, cardio-vibrational data samples 424a-n), physiological metrics 414a-n, and/or clinical information 418a-n to assist the medical professional or clinician in analyzing the information.

In operation, in some embodiments, as physiological data sets 406 are collected from various patients of the patient population 408a-n, the ECG and/or cardio-vibrational data sets 420a-n, 422a-n, 424a-n are analyzed by the machine learning engine 402a and cardiac condition measures 404a-n are collected in the historic cardiac condition measures 426a-n. Upon request (e.g., opening of a clinician and/or physician portal) or periodically (e.g., once per hour, once per day, once per week, etc.), the cardiac condition measure comparing engine 428 calculates the improvement scores 434a-n and provides the improvement scores 434a-n to the report generator 430 for generating the presentation 432 for the medical professional or clinician. Other real-time, near real-time, or batch processing flows may be applied while remaining in the scope and spirit of the process flow 400.

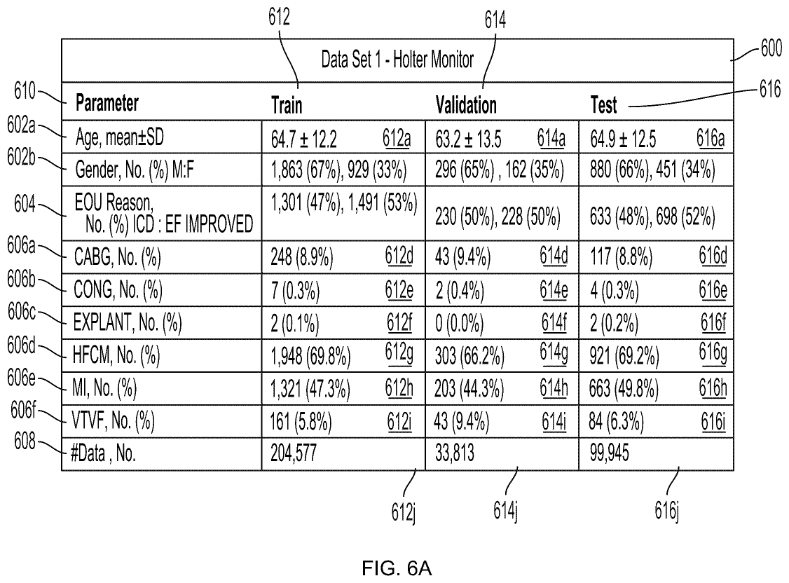

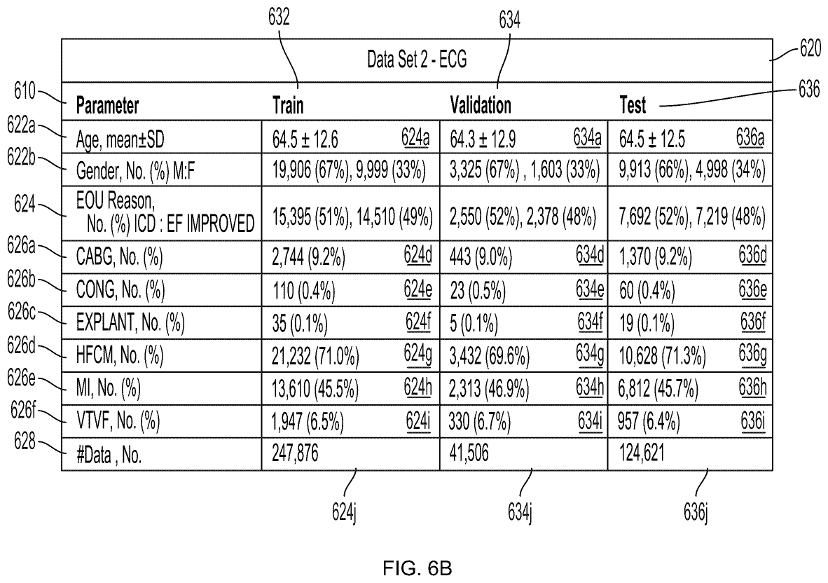

To test the accuracy of the multi-tier machine learning analysis as described above, the inventors obtained and labeled three different data sets: holter monitor data (FIG. 6A), LifeVest® WCD ECG data (FIG. 6B), and LifeVest® WCD cardio-vibrational data (FIG. 6A) corresponding to a portion of the LifeVest® WCD ECG data. Below is a discussion on the preparation of the data, machine learning model training, and results obtained through testing. As illustrated by the data presented below, the two-tier convolutional neural network model including a convolutional tier and a fully connected tier has been demonstrated to achieve, in initial testing, an area under the curve (AUC) performance of the machine learning engine including results of at least 70% in certain test scenarios, between about 70% and about 75% in certain test scenarios, between about 75% and about 80% in certain test scenarios, and between about 80% and about 85% in certain test scenarios.

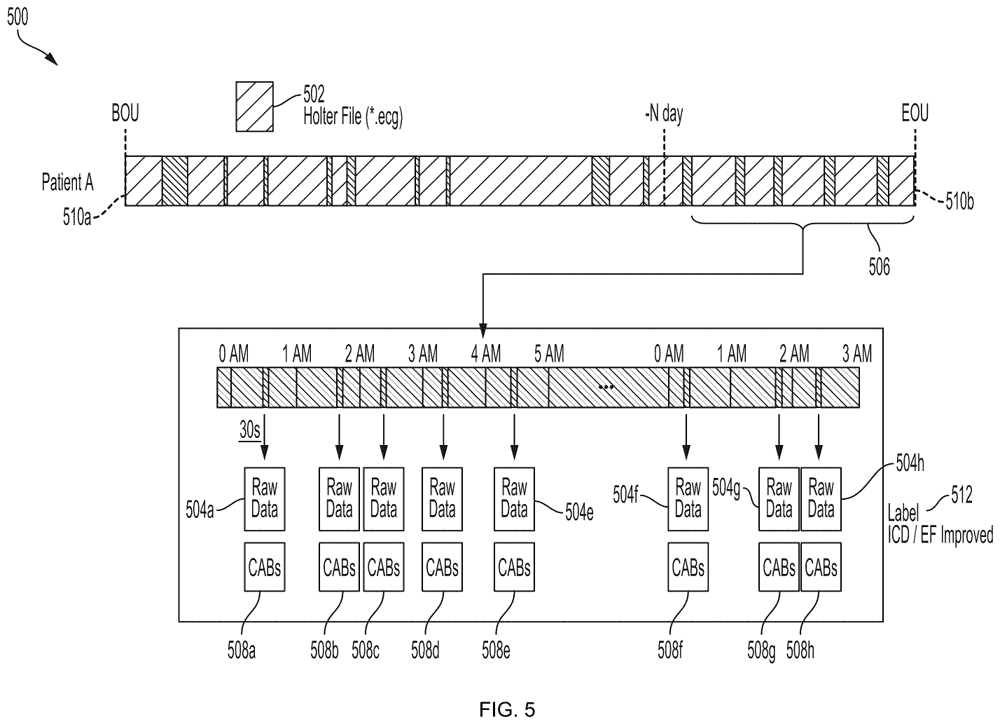

As illustrated in FIG. 5, the inventors obtained a first data set (“data set 1” 600, FIG. 6A) by extracting 30-second data samples 504 of ECG and cardio-vibrational data 502. The data samples 504 were collected by wearable Holter monitors over a period of time including multiple days of monitoring, beginning at a beginning of use (BOU) time 510a and ending at an end of use (EOU) time 510b. As illustrated, for example, some of the data samples were accessed periodically throughout a final day 506 of wearing the Holter monitor. Each data sample 504a-h includes corresponding cardiac acoustic biomarkers (CABs) 508a-h (EMAT, EMATc, SDI, S3, S4, LVST, and heart rate). The CABs 508a-h were calculated using a cardio-vibrational data portion of the corresponding data sample 504a-h. The CABs 508 are examples of physiological metrics, such as the metrics 114 described in relation to FIG. 1A and FIG. 1B and/or the processed data sets 412 of FIG. 4.

Turning to FIG. 6A, the following clinical information 606 (e.g., patient diagnoses and past medical procedures) was obtained regarding each patient of the holter monitor data set 600: indication of a coronary artery bypass grafting (CABG) procedure on the patient 606a, an indication of a congenital heart defect (CONG) diagnosis of the patient 606b, an indication of an implantable cardioverter defibrillator explant (EXPLANT) 606c, an indication of heart failure or cardiomyopathy (HFCM) in the patient 606d, an indication of diagnosis of a prior myocardial infarction (MI) of the patient 606e, and an indication of prior ventricular tachycardia (VTVF) of the patient 606f Further, each data sample included the corresponding patient demographic information of age 602a and gender 602b. The patient demographic information 602 and patient diagnoses and past medical procedures information 606 are examples of patient information 116 as described in relation to FIG. 1A and FIG. 1B and/or the patient information data sets 416 of FIG. 4.

The first data set 600 was labeled based upon the recorded reason for ending use of the Holter device out of two potential end of use reasons 604: ejection fraction (EF) improved or patient received an implantable cardioverter defibrillator (ICD) due to worsening EF (see also label 512 of FIG. 5). The ejection fraction, as referenced in relation to the validation and testing of the models described below, relates to the left ventricular ejection fraction (LVEF). However, in other embodiments, the methods and systems described herein may be applied to determining any or all of the LVEF, the right ventricular ejection fraction (RVEF), the left atrial ejection fraction (LAEF), and the right atrial ejection fraction (RAEF). As illustrated in a training column 612, a validation column 614, and a testing column 616 of the first data set 600, the end of use reason 604 was fairly evenly distributed between EF improved and ICD implant (e.g., 47%:53% ICD:EF Improved in the training data 612).

Similarly, turning to FIG. 6B, the LifeVest® WCD ECG data set (“Data set 2”) 620 includes the same parameters 610 (e.g., patient demographic information 622 and patient clinical information 626). The data has also been labeled based upon the recorded reason for ending use of the LifeVest® WCD device out of two potential end of use reasons 624 of ejection fraction (EF) improved or patient received an implantable cardioverter defibrillator (ICD) due to worsening EF. Further, the data has been labeled based on recorded left ventricular ejection fraction (LVEF) measurements measured at both the beginning of use and the end of use. Here, again, the end of use reason 624 was fairly evenly distributed between EF improved and ICD implant (e.g., 51% to 49% ICD to EF ratio Improved in the training data 632 of the data set 2 620). Data samples 628 represent thirty-second raw ECG data samples accessed from data collected by ECG sensors of a LifeVest® WCD on a final day of patient wear, similar to the process 500 described in relation to the holter data file 502 of FIG. 5.

To train the models for determining the ejection fraction of a patient, the inventors trained seven deep learning models with the raw sample data (ECG or ECG and cardio-vibrational data) of data set 600 of FIG. 6A and data set 620 of FIG. 6B along with the associated cardiac acoustic biomarkers (CABs), demographic information, and clinical information in various combinations. The inventors also trained two machine learning models using the CABs, clinical information, and demographic information of data set 1 600. For each model, the output was designed to categorize the ejection fraction as EF improved (e.g., the patient ceased wearing the cardiac monitoring device because of an improved cardiac condition) or ICD (e.g., the patient ceased wearing the cardiac monitoring device because of receiving an implantable cardiac defibrillator). These output classifications correspond to the data labels 512 and 604 discussed in relation to FIG. 5, FIG. 6A, and FIG. 6B. Thus, the output classifications are aligned to support validation of the model training through application of the data set 600 and data set 620.

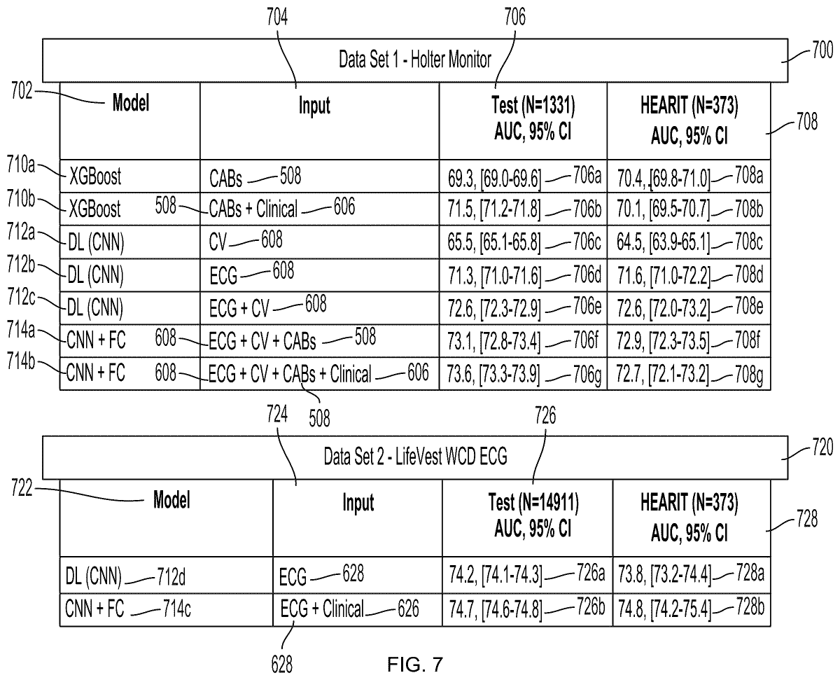

For initial machine learning models, turning to FIG. 7, the inventors trained a first XGBoost model 710a with the CABs 508 identified in FIG. 5 and a second XGBoost model 710b with both the CABs 508 and the clinical information 606 of data set 1 600 of FIG. 6A. An XGBoost model applies a gradient boosting decision tree algorithm for machine learning predictions. The gradient boosting decision tree algorithm is appropriate to the inputs which are represented by simple numeric values. As illustrated in FIG. 6A, the data set 1 600 included 207,577 data samples 612, corresponding to 2,792 patients.

The inventors also trained deep learning models, including convolutional neural network (CNN) models 712 as well as two-tier convolutional neural network models 714 having both a convolutional tier and a fully connected tier. The convolutional neural network algorithm is appropriate to the complex signal inputs (ECG data and cardio-vibrational data) fed into the models 712. The inventors trained the models 712 and 714 using both Holter monitor data (data set 1 600 of FIG. 6A) as well as LifeVest® WCD ECG data (data set 2 620 of FIG. 6B, including 257,876 samples 632, corresponding to 29,905 patients). The input fed into each separate model trained with holter monitor data 600 is identified in an input column 704 of a holter monitor training and testing table 700, and the input fed into each separate model trained with the LifeVest® WCD ECG data 620 is identified in an input column 724 of a LifeVest® WCD ECG training and testing table 720. For example, a first CNN model 712a was trained with a cardio-vibrational data portion of the data records 612j of FIG. 6A, a second CNN model 712b was trained with an ECG data portion of the data records 612j of the data set 1 of FIG. 6A, and a third CNN model 712c was trained with both the cardio-vibrational data portion and the ECG data portion of the data records 612j (e.g., at least two classifier stages including one or more cardio-vibrational data classifiers and one or more ECG data classifiers). The holter monitor data-trained models additionally include a first two-tier CNN model 714a trained with the ECG data portion of the data records 612j, the cardio-vibrational data portion of the data records 612j, and the CABs 508 as well as a second two-tier CNN model 714b trained with the ECG data portion of the data records 612j, the cardio-vibrational data portion of the data records 612j, the CABs 508, and the clinical information 606.

Turning to the LifeVest® WCD ECG training and testing table 720, a fourth CNN model 712d was trained with the ECG data portion of the data records 612j of data set 2 620 of FIG. 6B, and a third two-tier model 714c was trained with the ECG data portion of the data records 612j and the clinical information 626 of data set 2 620 of FIG. 6B.

The inventors validated the trained models 710, 712, and 714 using a subset 614j, 634j of the data records 608, 612j of the respective data sets 600, 620 of FIG. 6A and FIG. 6B as appropriate to the individual model's training, as illustrated in a validation column 614 of FIG. 6A and a validation column 634 of FIG. 6B. For example, the subset 614j corresponds to 458 out of 2,792 patients of the training data 604j, and the subset 634j corresponds to 4,928 out of 29,905 patients of the training data 624j.

Turning to FIG. 7, test results 706 for data set 1 600 and test results 726 for data set 2 620 are presented as area under the curve (AUC) performance calculations for each of the corresponding models 710, 712. The test results were obtained through application of test data records 616j of data set 1 600 (99,945 data samples corresponding to 1,331 patients) and test data records 636j of data set 2 620 (124,621 data samples corresponding to 14,911 patients). The AUC results represent the propensity of correct determinations in comparison to truth data (e.g., the measured ejection fraction or the ejection fraction category of the corresponding patient). The AUC values 706 and 726 each represent a percentage of correct determinations during the test phase. As illustrated, the multi-tier models 714a-c, including both raw signal data (ECG or combined ECG and cardio-vibrational data) and patient metrics (e.g. CABs and/or clinical data) performed better than raw data and/or patient metrics alone. Further, the two-tier model 714c tested against the larger patient population of the LifeVest® WCD ECG data set 620, demonstrated superior performance in comparison to the models trained using the smaller Holter monitor data set 600. The small pool of patient data available including cardio-vibrational samples may further contribute to this difference. In testing, the cardio-vibrational test data set included 37,246 data samples collected from 373 patients with many more male than female patients, as illustrated in the table below.

TABLE 1

| Parameter | Test |
|---|---|
| Age, mean ± SD | 61.7 ± 12.3 |
| Gender, No. (%) M:F | 289 (77%), 84 (23%) |
| EF Value, No. (%) EF <=35%:EF >35% | 214 (48%), 159 (52%) |
| #Data, No. | 37,246 |

Included in the AUC values 706 and 726 of FIG. 7 are corresponding confidence intervals, each represented as a range from a lower limit of the confidence interval (CI) to an upper limit of the CI. The CI range, as illustrated, is bounded by a set of brackets. The confidence interval represents, columns 706, 708, 726, and 728, a 95% confidence for the AUC being within the represented range (or interval) including the calculated AUC. As is shown in the data presented in columns 706, 708, 726, and 728, the CI ranges corresponding to the XGBoost models 710 and the CNN models 712 do not overlap the CI ranges associated with the two-tier convolutional models 714 for the same corresponding data set. Thus, the two-tier convolutional models 714 represent a discrete improvement upon applying the XGBoost models 710 or the CNN models 712. For example, turning to data set 2 720, an upper limit of the AUC CI range 726a corresponding to the CNN model 712d analyzing ECG data 628 alone is 74.3, while a lower limit of the AUC CI range 726b corresponding to two-tier convolutional model 714c analyzing both ECG data 628 and clinical data 626 is 74.6.

Table 2, below, illustrates, for two-tier model 714c applied to data set 2 620, a break-down of area under the curve results (e.g., AUC 74.8 728b of FIG. 7) by a day versus night distribution of cardiac measure determinations. As illustrated, a comparison of data collected during a daytime period (e.g., 10:00 AM to 10:00 PM) versus a night time period (e.g., midnight to 6:00 AM) resulted in a differing AUC of 76.5:72.8.

TABLE 2

| Input | AUC |
|---|---|
| Day Time (Time: 10-18) | 76.5 |
| Night Time (Time: 0-6) | 72.8 |

The inventors further tested models using registry data (“Data Set 3”) including the following information represented in Table 3. The data encompasses 373 patients and a total data sample set of 37,246 samples. Results of testing using the data represented in Table 3 are presented in a HEARIT AUC column 708 of data set 1 700 of FIG. 7 and column 728 of data set 2 720 of FIG. 7.

TABLE 3

| Parameter | Test |
|---|---|
| Age, mean ± SD | 61.7 ± 12.3 |
| Gender, No. (%) M:F | 289 (77%), 84 (23%) |
| EF Value, No. (%) EF <=35%:EF >35% | 214 (48%), 159 (52%) |
| Ischemic, No. (%) | 179 (48.0%) |
| Non-Ischemic, No. (%) | 169 (45.3%) |
| Mixed, No. (%) | 23 (6.2%) |
| #Data, No. | 37,246 |

Table 4, below, illustrates, for two-tier model 714c applied to data set 2 620, a break-down of area under the curve results 728b (74.8) by diagnostic classification of cardiac measure determinations. The diagnoses include ischemic (179 patients), non-ischemic (169 patients) and mixed (23 patients). Ischemia generally relates to blood flow restriction. In terms of data set 2 620, ischemic patients are those exhibiting blood flow restriction in the coronary arteries. The mixed designation pertains to patients that are registered in the data records as having moved over time from ischemic to non-ischemic or vice-versa, for example based on an event or an outcome of clinical testing and/or imaging. As illustrated, an area under the curve corresponding to the ischemic patients was 79.9, an AUC corresponding to the non-ischemic patients was 67.8, and an AUC corresponding to the mixed patients was 88.1, demonstrating a high rate of correct determinations corresponding to patients having been diagnosed with ischemia at least at some point before or during device use.

TABLE 4

| Input | AUC |
|---|---|
| Ischemic (N = 179) | 79.9 |
| Non-Ischemic (N = 169) | 67.8 |
| Mixed (N = 23) | 88.1 |

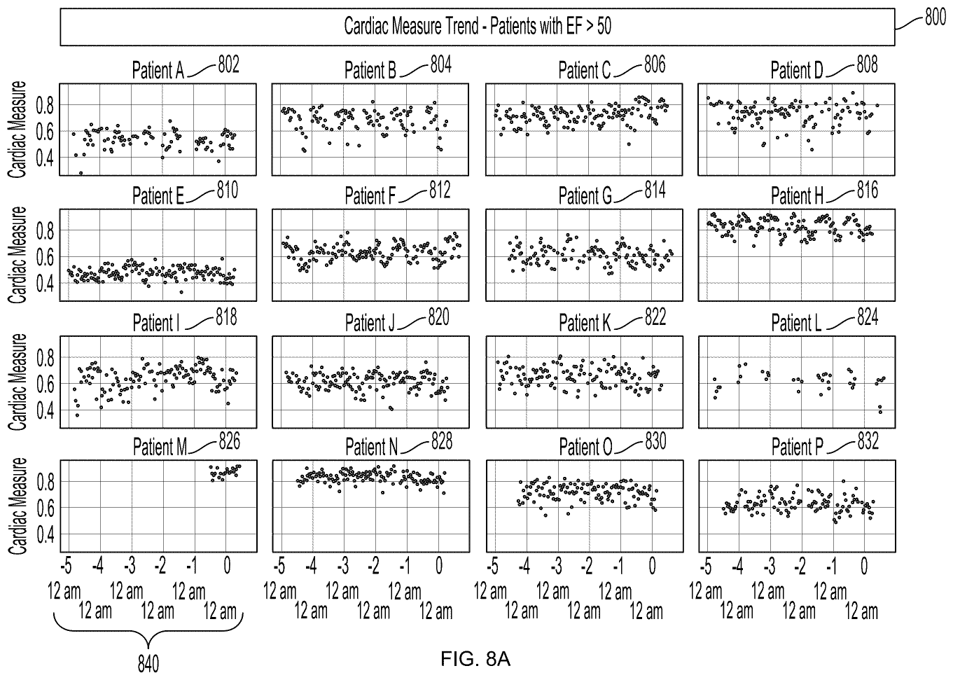

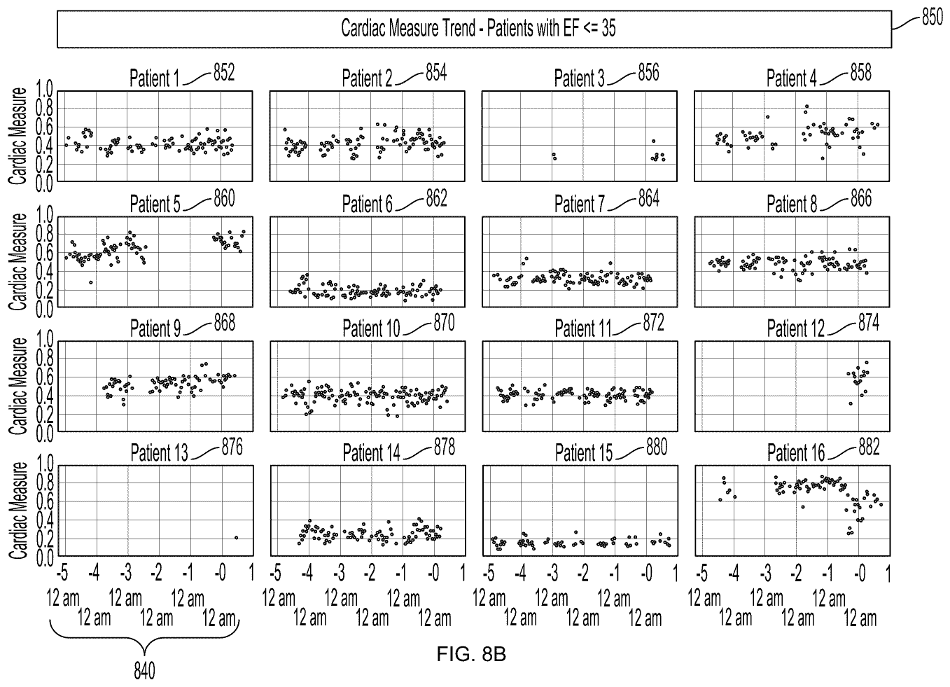

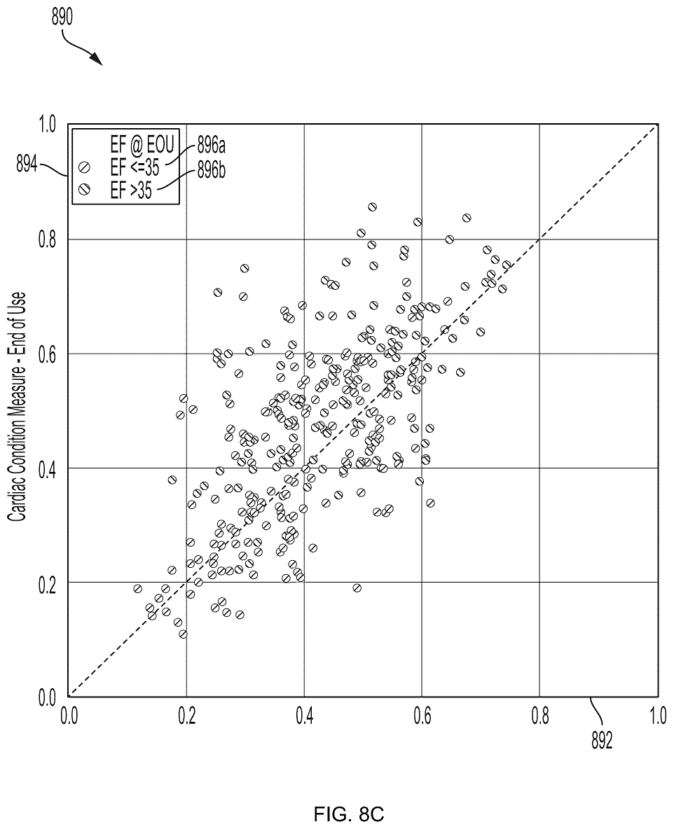

FIG. 8A and FIG. 8B illustrate graphical analysis is trends of cardiac measure determinations over time for sets of patients. The data presented in the graphs of FIG. 8A and FIG. 8B was obtained by applying data set 2 620 of FIG. 6B to two-tier model 714c of FIG. 7. Turning to FIG. 8A, a set of graphs 800 illustrate cardiac measure trends over a span of time 840 including five consecutive days of analysis for a patient population having an ejection fraction greater than 50 (e.g., a healthy ejection fraction). Conversely, turning to FIG. 8B, a set of graphs 850 illustrate cardiac measure trends over a span of time 840 including five consecutive days of analysis for a patient population having an ejection fraction less than or equal to 35 (e.g., a low ejection fraction). As illustrated, while hourly outcomes vary, the trends are generally stable across a period of time. As illustrated, greater accuracy may be obtained by sampling cardiac measure determinations and combining the samples across a period of time such as an hour or day.

FIG. 8C illustrates a graphical distribution 890 of ejection fraction determinations including determinations made using data from a beginning of use period of time 892 in comparison to data from an end of use period of time 894. The ejection fraction determinations were made by applying data set 2 620 of FIG. 6B to the two-tier model 714c of FIG. 7. The data is further characterized as corresponding to an ejection fraction (EF) of either less than or equal to 35 896a or greater than 35 896_{b}. As illustrated by the distribution, many outcomes were substantially identical, while some demonstrated significant change.

Table 5, below, illustrates performance parameters of the two-tier machine learning test model 714c of FIG. 7 when applying the HEARIT data of Table 3 (above). Table 5 demonstrates performance data for parameters including specificity (true positive÷(true positive+false negative), sensitivity (true positive÷(true positive+false negative), positive predictive value (PPV) and negative predictive value (NPV).

TABLE 5

| Specificity | Sensitivity | PPV | NPV |
|---|---|---|---|
| 0.2 | 0.968 | 0.598 | 0.835 |
| 0.6 | 0.750 | 0.697 | 0.662 |
| 0.8 | 0.532 | 0.766 | 0.582 |
| 0.9 | 0.384 | 0.825 | 0.543 |

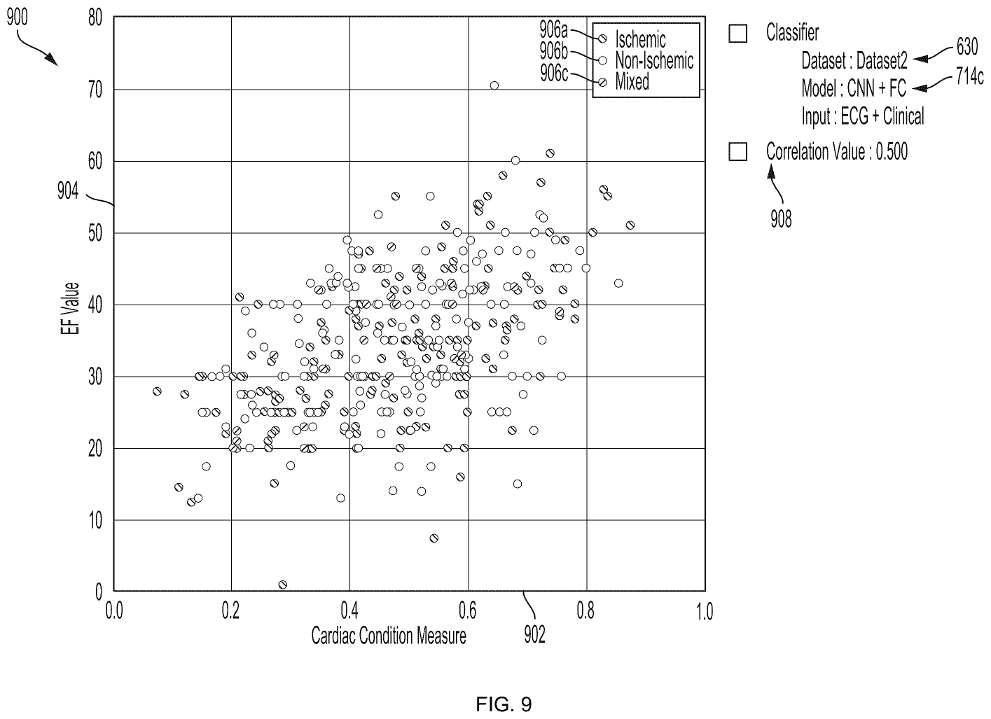

FIG. 9 illustrates a graphical distribution 900 of determined cardiac condition measures 902 graphed against ejection fraction values 904. The data points 906 are divided into diagnostic groupings of ischemic 906a, non-ischemic 906b and mixed 906c. The determinations to actual values within this test demonstrate a 0.500 (e.g., moderate) correlation value.

The teachings of the present disclosure can be generally applied to external medical monitoring and/or treatment devices that include one or more sensors as described herein. Such external medical devices can include, for example, ambulatory medical devices as described herein that are capable of and designed for moving with the patient as the patient goes about his or her daily routine. An example ambulatory medical device can be a wearable medical device such as a wearable cardioverter defibrillator (WCD), an in-hospital device such as an in-hospital wearable defibrillator (HWD), a short-term wearable cardiac monitoring and/or therapeutic device, mobile cardiac event monitoring devices, and other similar wearable medical devices.

The wearable medical device can be capable of continuous use by the patient. In some implementations, the continuous use can be substantially or nearly continuous in nature. That is, the wearable medical device can be continuously used, except for sporadic periods during which the use temporarily ceases (e.g., while the patient bathes, while the patient is refit with a new and/or a different garment, while the battery is charged/changed, while the garment is laundered, etc.). Such substantially or nearly continuous use as described herein may nonetheless be considered continuous use. For example, the wearable medical device can be configured to be worn by a patient for as many as twenty-four hours a day. In some implementations, the patient can remove the wearable medical device for a short portion of the day (e.g., for half an hour to bathe).

Further, the wearable medical device can be configured as a long term or extended use medical device. Such devices can be configured to be used by the patient for an extended period of several days, weeks, months, or even years. In some examples, the wearable medical device can be used by a patient for an extended period of at least one week. In some examples, the wearable medical device can be used by a patient for an extended period of at least 30 days. In some examples, the wearable medical device can be used by a patient for an extended period of at least one month. In some examples, the wearable medical device can be used by a patient for an extended period of at least two months. In some examples, the wearable medical device can be used by a patient for an extended period of at least three months. In some examples, the wearable medical device can be used by a patient for an extended period of at least six months. In some examples, the wearable medical device can be used by a patient for an extended period of at least one year. In some implementations, the extended use can be uninterrupted until a physician or other health care practitioner (HCP) provides specific instruction to the patient to stop use of the wearable medical device.

Regardless of the extended period of wear, the use of the wearable medical device can include continuous or nearly continuous wear by the patient as described above. For example, the continuous use can include continuous wear or attachment of the wearable medical device to the patient, e.g., through one or more of the electrodes as described herein, during both periods of monitoring and periods when the device may not be monitoring the patient but is otherwise still worn by or otherwise attached to the patient. The wearable medical device can be configured to continuously monitor the patient for cardiac-related information (e.g., ECG information, including arrhythmia information, cardio-vibrations, etc.) and/or non-cardiac information (e.g., blood oxygen, the patient's temperature, glucose levels, tissue fluid levels, and/or lung vibrations). The wearable medical device can carry out its monitoring in periodic or aperiodic time intervals or times. For example, the monitoring during intervals or times can be triggered by a user action or another event.

As noted above, the wearable medical device can be configured to monitor other non-ECG physiologic parameters of the patient in addition to cardiac related parameters. For example, the wearable medical device can be configured to monitor, for example, pulmonary-vibrations (e.g., using microphones and/or accelerometers), breath vibrations, sleep related parameters (e.g., snoring, sleep apnea), tissue fluids (e.g., using radio-frequency transmitters and sensors), among others.

Other example wearable medical devices include automated cardiac monitors and/or defibrillators for use in certain specialized conditions and/or environments such as in combat zones or within emergency vehicles. Such devices can be configured so that they can be used immediately (or substantially immediately) in a life-saving emergency. In some examples, the ambulatory medical devices described herein can be pacing-enabled, e.g., capable of providing therapeutic pacing pulses to the patient. In some examples, the ambulatory medical devices can be configured to monitor for and/or measure ECG metrics including, for example, heart rate (such as average, median, mode, or other statistical measure of the heart rate, and/or maximum, minimum, resting, pre-exercise, and post-exercise heart rate values and/or ranges), heart rate variability metrics, PVC burden or counts, atrial fibrillation burden metrics, pauses, heart rate turbulence, QRS height, QRS width, changes in a size or shape of morphology of the ECG information, cosine R-T, artificial pacing, QT interval, QT variability, T wave width, T wave alternans, T-wave variability, and ST segment changes.

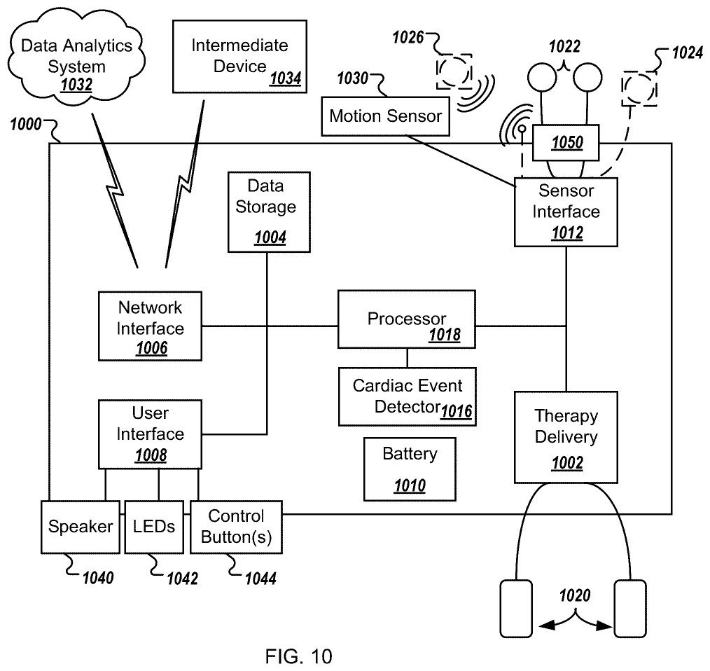

FIG. 10 illustrates an example component-level view of a medical device controller 1000 included in, for example, a wearable medical device. As further shown in FIG. 10, the therapy delivery circuitry 1002 can be coupled to one or more electrodes 1020 configured to provide therapy to the patient. For example, the therapy delivery circuitry 1002 can include, or be operably connected to, circuitry components that are configured to generate and provide an electrical therapeutic shock. The circuitry components can include, for example, resistors, capacitors, relays and/or switches, electrical bridges such as an h-bridge (e.g., including a number of insulated gate bipolar transistors or IGBTs), voltage and/or current measuring components, and other similar circuitry components arranged and connected such that the circuitry components work in concert with the therapy delivery circuitry and under control of one or more processors (e.g., processor 1018) to provide, for example, at least one therapeutic shock to the patient including one or more pacing, cardioversion, or defibrillation therapeutic pulses.

Pacing pulses can be used to treat cardiac arrhythmia conditions such as bradycardia (e.g., less than 30 beats per minute) and tachycardia (e.g., more than 150 beats per minute) using, for example, fixed rate pacing, demand pacing, anti-tachycardia pacing, and the like. Defibrillation shocks can be used to treat ventricular tachycardia and/or ventricular fibrillation.

For example, each defibrillation shock can deliver between 60 to 180 joules of energy. In some implementations, the defibrillating shock can be a biphasic truncated exponential waveform, whereby the signal can switch between a positive and a negative portion (e.g., charge directions). This type of waveform can be effective at defibrillating patients at lower energy levels when compared to other types of defibrillation shocks (e.g., such as monophasic shocks). For example, an amplitude and a width of the two phases of the energy waveform can be automatically adjusted to deliver a precise energy amount (e.g., 150 joules) regardless of the patient's body impedance. The therapy delivery circuitry 1002 can be configured to perform the switching and pulse delivery operations, e.g., under control of the processor 1018. As the energy is delivered to the patient, the amount of energy being delivered can be tracked. For example, the amount of energy can be kept to a predetermined constant value even as the pulse waveform is dynamically controlled based on factors such as the patient's body impedance which the pulse is being delivered.

In certain examples, the therapy delivery circuitry 1002 can be configured to deliver a set of cardioversion pulses to correct, for example, an improperly beating heart. When compared to defibrillation as described above, cardioversion typically includes a less powerful shock that is delivered at a certain frequency to mimic a heart's normal rhythm.

A data storage region 1004 can include one or more of non-transitory computer-readable media, such as flash memory, solid state memory, magnetic memory, optical memory, cache memory, combinations thereof, and others. The data storage 1004 can be configured to store executable instructions and data used for operation of the medical device controller 1000. In certain examples, the data storage 1004 can include executable instructions that, when executed, are configured to cause the processor 1018 to perform one or more operations. In some examples, the data storage 1004 can be configured to store information such as ECG data as received from, for example, a sensing electrode interface 1012.

In some embodiments, a network interface 1006 can facilitate the communication of information between the medical device controller 1000 and one or more other devices or entities over a communications network. For example, where the medical device controller 1000 is included in an ambulatory medical device, the network interface 1006 can be configured to communicate with a remote computing device such as a remote server or other similar computing device. In further embodiments, the remote computing device can be part of a remote data analytics system 1032. The network interface 1006 can include communications circuitry for transmitting data in accordance with a Bluetooth® or Zigbee® wireless standard for exchanging such data over short distances to an intermediary device 1034. In some examples, the intermediary device 1034 can be configured as a base station, a “hotspot” device, a smartphone, a tablet, a portable computing device, and/or other devices in proximity of the wearable medical device including the medical device controller 1000. The intermediary device(s) 1034 may in turn communicate the data to a remote server over a broadband cellular network communications link, such as the data analytics system 1032. The communications link may implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the intermediary device(s) 1034 may communicate with a remote server over a Wi-Fi® communications link based on the IEEE 802.11 standard.

In certain embodiments, a user interface 1008 can include one or more physical interface devices such as input devices, output devices, and combination input/output devices and a software stack configured to drive operation of the devices. These user interface elements can render visual (e.g. LEDs 1042), audio (e.g., speaker 1040), and/or tactile content. Thus, the user interface 1008 can receive input (e.g., via one or more control buttons 1044) or provide output, thereby enabling a user to interact with the medical device controller 1000.

The medical device controller 1000, in some embodiments, includes at least one power source (e.g., rechargeable battery) 1010 configured to provide power to one or more components integrated in the medical device controller 1000. The battery 1010 can include a rechargeable multi-cell battery pack. In one example implementation, the battery 1010 can include three or more 2200 mAh lithium ion cells that provide electrical power to the other device components within the medical device controller 1000. For example, the battery 1010 can provide its power output in a range of between 20 mA to 1000 mA (e.g., 40 mA) output and can support 24 hours, 48 hours, 72 hours, or more, of runtime between charges. In certain implementations, the battery capacity, runtime, and type (e.g., lithium ion, nickel-cadmium, or nickel-metal hydride) can be changed to best fit the specific application of the medical device controller 1000.

A sensor interface 1012, in some embodiments, includes physiological signal circuitry that is coupled to one or more sensors configured to monitor one or more physiological parameters of the patient. As shown, the sensors 1022, 1024, 1026, 1030 can be coupled to the medical device controller 1000 via a wired or wireless connection. The sensors can include one or more ECG sensing electrodes 1022 and non-ECG physiological sensors such as a vibration sensor 1024, tissue fluid monitor(s) 1026 (e.g., based on ultra-wide band RF devices), and motion sensor(s) 1030 (e.g., accelerometers, gyroscopes, and/or magnetometers). In some implementations, the sensors can include a number of conventional ECG sensing electrodes 1022 in addition to digital sensing electrodes 1022.

The sensing electrodes 1022 can be configured to monitor a patient's ECG information. For example, by design, the digital sensing electrodes 1022 can include skin-contacting electrode surfaces that may be deemed polarizable or non-polarizable depending on a variety of factors including the metals and/or coatings used in constructing the electrode surface. All such electrodes can be used with the principles, techniques, devices and systems described herein. For example, the electrode surfaces can be based on stainless steel, noble metals such as platinum, or Ag—AgCl.

In some examples, the electrodes 1022 can be used with an electrolytic gel dispersed between the electrode surface and the patient's skin. In certain implementations, the electrodes 1022 can be dry electrodes that do not need an electrolytic material. As an example, such a dry electrode can be based on tantalum metal and having a tantalum pentoxide coating as is described above. Such dry electrodes can be more comfortable for long term monitoring applications.

The vibration sensor(s) 1024, in some implementations, can be configured to detect cardiac or pulmonary vibration information. For example, the vibration sensor(s) 1024 can detect a patient's heart valve vibration information. For example, the vibration sensor(s) 1024 can be configured to detect cardio-vibrational signal values including any one or all of S1, S2, S3, and S4. From these cardio-vibrational signal values or heart vibration values, certain heart vibration metrics may be calculated, including any one or more of electromechanical activation time (EMAT), average EMAT, percentage of EMAT (% EMAT), systolic dysfunction index (SDI), and left ventricular systolic time (LVST). The vibration sensor(s) 1024 can also be configured to detect heart wall motion, for instance, by placement of the sensor in the region of the apical beat. The vibration sensor(s) 1024 can include a vibrational sensor configured to detect vibrations from a subject's cardiac and pulmonary system and provide an output signal responsive to the detected vibrations of a targeted organ, for example, being able to detect vibrations generated in the trachea or lungs due to the flow of air during breathing. In certain implementations, additional physiological information can be determined from pulmonary-vibrational signals such as, for example, lung vibration characteristics based on pulmonary vibrations produced within the lungs (e.g., stridor, crackle, etc.). The vibration sensor(s) 1024 can also include a multi-channel accelerometer, for example, a three-channel accelerometer configured to sense movement in each of three orthogonal axes such that patient movement/body position can be detected and correlated to detected cardio-vibrations information. The vibration sensor(s) 1024 can transmit information descriptive of the cardio-vibrations information to the sensor interface 1012 for subsequent analysis.

The tissue fluid monitor(s) 1026 can use radio frequency (RF) based techniques to assess fluid levels and accumulation in a patient's body tissue. For example, the tissue fluid monitor(s) 1026 can be configured to measure fluid content in the lungs, typically for diagnosis and follow-up of pulmonary edema or lung congestion in heart failure patients. The tissue fluid monitor(s) 1026 can include one or more antennas configured to direct RF waves through a patient's tissue and measure output RF signals in response to the waves that have passed through the tissue. In certain implementations, the output RF signals include parameters indicative of a fluid level in the patient's tissue. The tissue fluid monitor(s) 1026 can transmit information descriptive of the tissue fluid levels to the sensor interface 1012 for subsequent analysis.

In certain implementations, a cardiac event detector 1016 can be configured to monitor a patient's ECG signal for an occurrence of a cardiac event such as an arrhythmia or other similar cardiac event. The cardiac event detector 1016 can be configured to operate in concert with the processor 1018 to execute one or more methods that process received ECG signals from, for example, the sensing electrodes 1022 and determine the likelihood that a patient is experiencing a cardiac event. The cardiac event detector 1016 can be implemented using hardware or a combination of hardware and software. For instance, in some examples, cardiac event detector 1016 can be implemented as a software component that is stored within the data storage 1004 and executed by the processor 1018. In this example, the instructions included in the cardiac event detector 1016 can cause the processor 1018 to perform one or more methods for analyzing a received ECG signal to determine whether an adverse cardiac event is occurring. In other examples, the cardiac event detector 1016 can be an application-specific integrated circuit (ASIC) that is coupled to the processor 1018 and configured to monitor ECG signals for adverse cardiac event occurrences. Thus, examples of the cardiac event detector 1016 are not limited to a particular hardware or software implementation.

In some implementations, the processor 1018 includes one or more processors (or one or more processor cores) that each are configured to perform a series of instructions that result in manipulated data and/or control the operation of the other components of the medical device controller 1000. In some implementations, when executing a specific process (e.g., cardiac monitoring), the processor 1018 can be configured to make specific logic-based determinations based on input data received and be further configured to provide one or more outputs that can be used to control or otherwise inform subsequent processing to be carried out by the processor 1018 and/or other processors or circuitry with which processor 1018 is communicatively coupled. Thus, the processor 1018 reacts to specific input stimulus in a specific way and generates a corresponding output based on that input stimulus. In some example cases, the processor 1018 can proceed through a sequence of logical transitions in which various internal register states and/or other bit cell states internal or external to the processor 1018 can be set to logic high or logic low. As referred to herein, the processor 1018 can be configured to execute a function where software is stored in a data store coupled to the processor 1018, the software being configured to cause the processor 1018 to proceed through a sequence of various logic decisions that result in the function being executed. The various components that are described herein as being executable by the processor 1018 can be implemented in various forms of specialized hardware, software, or a combination thereof. For example, the processor 1018 can be a digital signal processor (DSP) such as a 24-bit DSP. The processor 1018 can be a multi-core processor, e.g., having two or more processing cores. The processor 1018 can be an Advanced RISC Machine (ARM) processor such as a 32-bit ARM processor or a 64-bit ARM processor. The processor 1018 can execute an embedded operating system, and include services provided by the operating system that can be used for file system manipulation, display & audio generation, basic networking, firewalling, data encryption and communications.

As noted above, an ambulatory medical device such as a WCD can be designed to include a digital front-end where analog signals sensed by skin-contacting electrode surfaces of a set of digital sensing electrodes are converted to digital signals for processing. Typical ambulatory medical devices with analog front-end configurations use circuitry to accommodate a signal from a high source impedance from the sensing electrode (e.g., having an internal impedance range from approximately 100 Kiloohms to one or more Megaohms). This high source impedance signal is processed and transmitted to a monitoring device such as processor 1018 of the controller 1000 as described above for further processing. In certain implementations, the monitoring device, or another similar processor such as a microprocessor or another dedicated processor operably coupled to the sensing electrodes, can be configured to receive a common noise signal from each of the sensing electrodes, sum the common noise signals, invert the summed common noise signals and feed the inverted signal back into the patient as a driven ground using, for example, a driven right leg circuit to cancel out common mode signals.

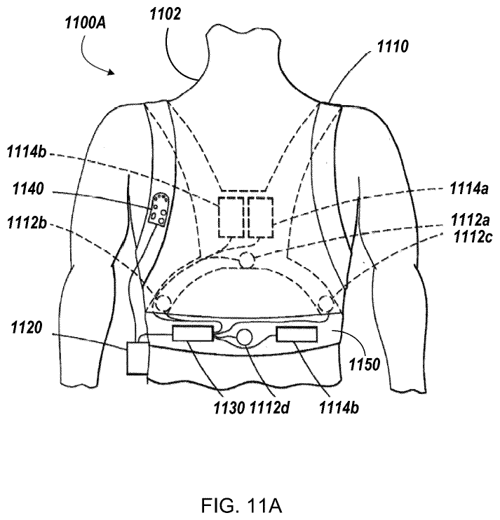

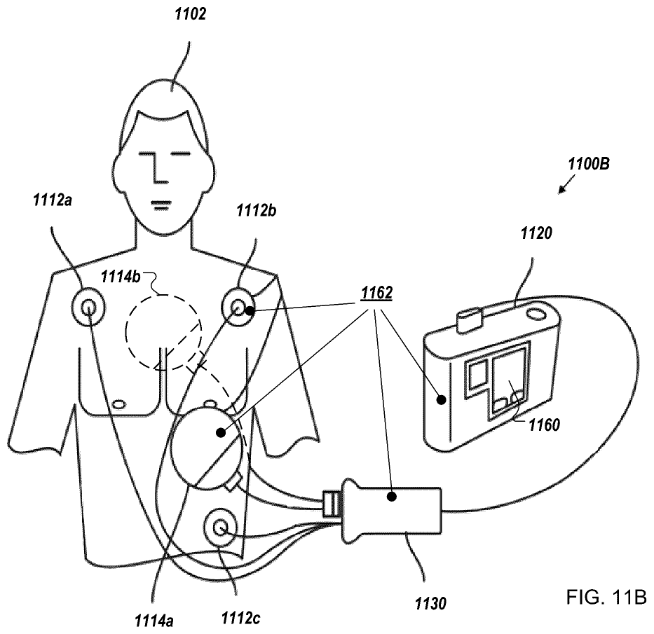

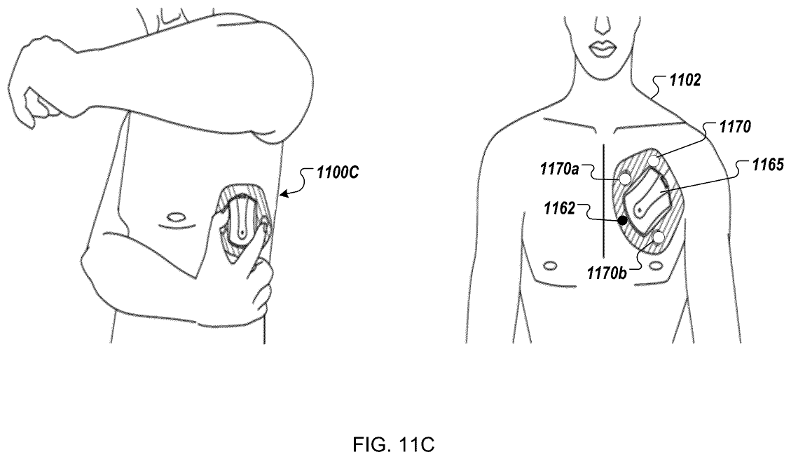

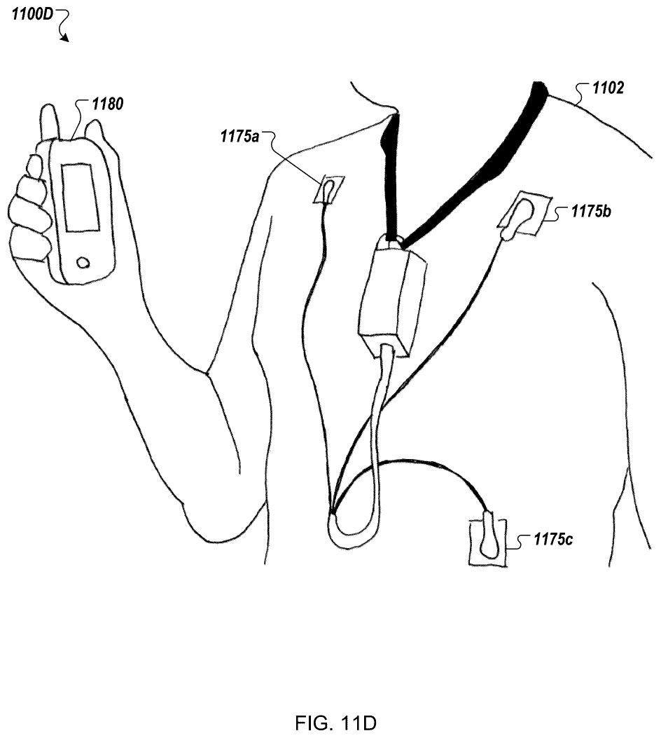

FIG. 11A illustrates an example medical device 1100 that is external, ambulatory, and wearable by a patient 1102, and configured to implement one or more configurations described herein. For example, the medical device 1100 can be a non-invasive medical device configured to be located substantially external to the patient. Such a medical device 1100 can be, for example, an ambulatory medical device that is capable of and designed for moving with the patient as the patient goes about his or her daily routine. For example, the medical device 1100 as described herein can be bodily-attached to the patient such as the LifeVest® wearable cardioverter defibrillator available from ZOLL® Medical Corporation. Such wearable defibrillators typically are worn nearly continuously or substantially continuously for two to three months at a time. During the period of time in which they are worn by the patient, the wearable defibrillator can be configured to continuously or substantially continuously monitor the vital signs of the patient and, upon determination that treatment is required, can be configured to deliver one or more therapeutic electrical pulses to the patient. For example, such therapeutic shocks can be pacing, defibrillation, or transcutaneous electrical nerve stimulation (TENS) pulses.

The medical device 1100 can include one or more of the following: a garment 1110, one or more ECG sensing electrodes 1112, one or more non-ECG physiological sensors such as the sensors 1024, 1026, 1030 described in relation to FIG. 10, one or more therapy electrodes 1114a and 1114b (collectively referred to herein as therapy electrodes 1114), a medical device controller 1120 (e.g., controller 1000 as described above in the discussion of FIG. 10), a connection pod 1130, a patient interface pod 1140, a belt 1150, or any combination of these. In some examples, at least some of the components of the medical device 1100 can be configured to be affixed to the garment 1110 (or in some examples, permanently integrated into the garment 1110), which can be worn about the patient's torso. In some implementations, at least a portion of the components of the medical device 1100 can be configured to be in wireless communication with other components of the medical device 1100. For example, the patient interface pod 1140 may be arranged as a remote control interface for use by the patient and in wireless communication with the medical device controller 1120.

The medical device controller 1120 can be operatively coupled to the sensing electrodes 1112, which can be affixed to the garment 1110, e.g., assembled into the garment 1110 or removably attached to the garment, for example using hook and loop fasteners, snaps, and/or Velcro. In some implementations, the sensing electrodes 1112 can be permanently integrated into the garment 1110. The medical device controller 1120 can be operatively coupled to the therapy electrodes 1114. For example, the therapy electrodes 1114 can also be assembled into the garment 1110, or, in some implementations, the therapy electrodes 1114 can be permanently integrated into the garment 1110. In an example, the medical device controller 1120 includes a patient user interface 1160 to allow a patient interface with the externally-worn device. For example, the patient can use the patient user interface 1160 to respond to activity related questions, prompts, and surveys as described herein.

Component configurations other than those shown in FIG. 11A are possible. For example, the sensing electrodes 1112 can be configured to be attached at various positions about the body of the patient 1102. The sensing electrodes 1112 can be operatively coupled to the medical device controller 1120 through the connection pod 1130. In some implementations, the sensing electrodes 1112 can be adhesively attached to the patient 1102. In some implementations, the sensing electrodes 1112 and at least one of the therapy electrodes 1114 can be included on a single integrated patch and adhesively applied to the patient's body.

The sensing electrodes 1112 can be configured to detect one or more cardiac signals. Examples of such signals include ECG signals and/or other sensed cardiac physiological signals from the patient. In certain examples, as described herein, the non-ECG physiological sensors 1113 such as accelerometers, vibrational sensors, RF-based sensors, and other measuring devices for recording additional non-ECG physiological parameters. For example, as described above, the non-ECG physiological sensors may be configured to detect other types of patient physiological parameters and acoustic signals, such as tissue fluid levels, cardio-vibrations, lung vibrations, respiration vibrations, and/or patient movement, etc.

In some examples, the therapy electrodes 1114 can also be configured to include sensors configured to detect ECG signals as well as other physiological signals of the patient. The connection pod 1130 can, in some examples, include a signal processor configured to amplify, filter, and digitize these cardiac signals prior to transmitting the cardiac signals to the medical device controller 1120. One or more of the therapy electrodes 1114 can be configured to deliver one or more therapeutic defibrillating shocks to the body of the patient 1102 when the medical device 1100 determines that such treatment is warranted based on the signals detected by the sensing electrodes 1112 and processed by the medical device controller 1120. Example therapy electrodes 1114 can include metal electrodes such as stainless-steel electrodes that include one or more conductive gel deployment devices configured to deliver conductive gel to the metal electrode prior to delivery of a therapeutic shock.

In some examples, the medical device 1100 can further includes one or more motion sensors such as accelerometers 1162. As shown in FIG. 11A, in some examples an accelerometer 1162 can be integrated into one or more of a sensing electrode 1112, a therapy electrode 1114, the medical device controller 1120, and various other components of the medical device 1100.

In some implementations, medical devices as described herein can be configured to switch between a therapeutic medical device and a monitoring medical device that is configured to only monitor a patient (e.g., not provide or perform any therapeutic functions). For example, therapeutic components such as the therapy electrodes 1114 and associated circuitry can be optionally decoupled from (or coupled to) or switched out of (or switched in to) the medical device 1100A. For example, a medical device can have optional therapeutic elements (e.g., defibrillation and/or pacing electrodes, components, and associated circuitry) that are configured to operate in a therapeutic mode. The optional therapeutic elements can be physically decoupled from the medical device to convert the therapeutic medical device into a monitoring medical device for a specific use (e.g., for operating in a monitoring-only mode) or a patient. Alternatively, the optional therapeutic elements can be deactivated (e.g., via a physical or a software switch), essentially rendering the therapeutic medical device as a monitoring medical device for a specific physiologic purpose or a particular patient. As an example of a software switch, an authorized person can access a protected user interface of the medical device and select a preconfigured option or perform some other user action via the user interface to deactivate the therapeutic elements of the medical device.

FIG. 11B illustrates a hospital wearable defibrillator 1100B that is external, ambulatory, and wearable by the patient 1102. Hospital wearable defibrillator 1100B can be configured in some implementations to provide pacing therapy, e.g., to treat bradycardia, tachycardia, and asystole conditions. The hospital wearable defibrillator 1100B can include one or more ECG sensing electrodes 1112, one or more therapy electrodes 1114, a medical device controller 1120 and a connection pod 1130. For example, each of these components can be structured and function as like number components of the medical device 1100A of FIG. 11A. For example, the electrodes 1112a-1112c, 1114a, 1114b can include disposable adhesive electrodes. For example, the electrodes 1112a, 1114a, 1114b can include sensing and therapy components disposed on separate sensing and therapy electrode adhesive patches.

In some implementations, both sensing and therapy components can be integrated and disposed on a same electrode adhesive patch that is then attached to the patient. For example, the front adhesively attachable therapy electrode 1114a attaches to the front of the patient's torso to deliver pacing or defibrillating therapy. Similarly, the back adhesively attachable therapy electrode 1114b attaches to the back of the patient's torso. In an example scenario, at least three ECG adhesively attachable sensing electrodes 1112a-1112c can be attached to at least above the patient's chest near the right arm, above the patient's chest near the left arm, and towards the bottom of the patient's chest in a manner prescribed by a trained professional.

A patient being monitored by a hospital wearable defibrillator and/or pacing device may be confined to a hospital bed or room for a significant amount of time (e.g., 75% or more of the patient's stay in the hospital). As a result, a user interface 1160 can be configured to interact with a user other than the patient, e.g., a nurse, for device-related functions such as initial device baselining, setting and adjusting patient parameters, and changing the device batteries.

In some examples, the hospital wearable defibrillator 1100B can further include one or more motion sensors such as accelerometers 1162. As shown in FIG. 11B, in some examples an accelerometer 1162 can be integrated into one or more of a sensing electrode 1112a (e.g., integrated into the same patch as the sensing electrode), a therapy electrode 1114a (e.g., integrated into the same patch as the therapy electrode), the medical device controller 1120, the connection pod 1130, and various other components of the hospital wearable defibrillator 1100B.

In some implementations, an example of a therapeutic medical device that includes a digital front-end in accordance with the systems and methods described herein can include a short-term defibrillator and/or pacing device. For example, such a short-term device can be prescribed by a physician for patients presenting with syncope. A wearable defibrillator can be configured to monitor patients presenting with syncope by, e.g., analyzing the patient's physiological and cardiac activity for aberrant patterns that can indicate abnormal physiological function. For example, such aberrant patterns can occur prior to, during, or after the onset of syncope. In such an example implementation of the short-term wearable defibrillator, the electrode assembly can be adhesively attached to the patient's skin and have a similar configuration as the hospital wearable defibrillator described above in connection with FIG. 11B.

FIGS. 11C and 11D illustrate example wearable patient monitoring devices with no treatment or therapy functions. For example, such devices are configured to monitor one or more physiological parameters of a patient, e.g., for remotely monitoring and/or diagnosing a condition of the patient. For example, such physiological parameters can include a patient's ECG information, tissue (e.g., lung) fluid levels, cardio-vibrations (e.g., using accelerometers or microphones), and other related cardiac information. A cardiac monitoring device is a portable device that the patient can carry around as he or she goes about their daily routine.

Referring to FIG. 11C, an example wearable patient monitoring device 1100C can include tissue fluid monitors 1165 that use RF based techniques to assess fluid levels and accumulation in a patient's body tissue. Such tissue fluid monitors 1165 can be configured to measure fluid content in the lungs, typically for diagnosis and follow-up of pulmonary edema or lung congestion in heart failure patients. The tissue fluid monitors 1165 can include one or more antennas configured to direct RF waves through a patient's tissue and measure output RF signals in response to the waves that have passed through the tissue. In certain implementations, the output RF signals include parameters indicative of a fluid level in the patient's tissue. In examples, device 1100C may be a cardiac monitoring device that also includes digital sensing electrodes 1170a, 1170b for sensing ECG activity of the patient. Device 1100C can pre-process the ECG signals via one or more ECG processing and/or conditioning circuits such as an ADC, operational amplifiers, digital filters, signal amplifiers under control of a microprocessor. Device 1100C can transmit information descriptive of the ECG activity and/or tissue fluid levels via a network interface to a remote server for analysis. Additionally, in certain implementations, the device 1100C can include one or accelerometers 1162 for measuring motion signals as described herein.

Referring to FIG. 11D, another example wearable cardiac monitoring device 1100D can be attached to a patient 1102 via at least three adhesive digital cardiac sensing electrodes 1175a-c disposed about the patient's torso. Additionally, in certain implementations, the device 1100D can include one or accelerometers integrated into, for example, one or more of the digital sensing electrodes for measuring motion signals as described herein.

Cardiac devices 1100C and 1100D are used in cardiac monitoring and telemetry and/or continuous cardiac event monitoring applications, e.g., in patient populations reporting irregular cardiac symptoms and/or conditions. These devices can transmit information descriptive of the ECG activity and/or tissue fluid levels via a network interface to a remote server for analysis. Example cardiac conditions that can be monitored include atrial fibrillation (AF), bradycardia, tachycardia, atrio-ventricular block, Lown-Ganong-Levine syndrome, atrial flutter, sino-atrial node dysfunction, cerebral ischemia, pause(s), and/or heart palpitations. For example, such patients may be prescribed a cardiac monitoring for an extended period of time, e.g., 10 to 30 days, or more. In some ambulatory cardiac monitoring and/or telemetry applications, a portable cardiac monitoring device can be configured to substantially continuously monitor the patient for a cardiac anomaly, and when such an anomaly is detected, the monitor can automatically send data relating to the anomaly to a remote server. The remote server may be located within a 24-hour manned monitoring center, where the data is interpreted by qualified, cardiac-trained reviewers and/or HCPs, and feedback provided to the patient and/or a designated HCP via detailed periodic or event-triggered reports. In certain cardiac event monitoring applications, the cardiac monitoring device is configured to allow the patient to manually press a button on the cardiac monitoring device to report a symptom. For example, a patient can report symptoms such as a skipped beat, shortness of breath, light headedness, racing heart rate, fatigue, fainting, chest discomfort, weakness, dizziness, and/or giddiness. The cardiac monitoring device can record predetermined physiologic parameters of the patient (e.g., ECG information) for a predetermined amount of time (e.g., 1-30 minutes before and 1-30 minutes after a reported symptom). As noted above, the cardiac monitoring device can be configured to monitor physiologic parameters of the patient other than cardiac related parameters. For example, the cardiac monitoring device can be configured to monitor, for example, cardio-vibrational signals (e.g., using accelerometers or microphones), pulmonary-vibrational signals, breath vibrations, sleep related parameters (e.g., snoring, sleep apnea), tissue fluids, among others.

In some examples, the devices described herein (e.g., FIGS. 11A-11D) can communicate with a remote server via an intermediary or gateway device 1180 such as that shown in FIG. 11D. For instance, devices such as shown in FIGS. 11A-D can be configured to include a network interface communications capability as described herein in reference to, for example, FIG. 10.

Additionally, the devices 1100A-D described herein in relation to FIGS. 11A-11D can be configured to include one or more vibrational sensors as described herein for collecting signals for use in producing cardio-vibrational image matrices.

Reference has been made to illustrations representing methods and systems according to implementations of this disclosure. Aspects thereof may be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/operations specified in the illustrations.

One or more processors can be utilized to implement various functions and/or algorithms described herein. Additionally, any functions and/or algorithms described herein can be performed upon one or more virtual processors, for example on one or more physical computing systems such as a computer farm or a cloud drive.

Aspects of the present disclosure may be implemented by hardware logic (where hardware logic naturally also includes any necessary signal wiring, memory elements and such), with such hardware logic able to operate without active software involvement beyond initial system configuration and any subsequent system reconfigurations (e.g., for different object schema dimensions). The hardware logic may be synthesized on a reprogrammable computing chip such as a field programmable gate array (FPGA) or other reconfigurable logic device. In addition, the hardware logic may be hard coded onto a custom microchip, such as an application-specific integrated circuit (ASIC). In other embodiments, software, stored as instructions to a non-transitory computer-readable medium such as a memory device, on-chip integrated memory unit, or other non-transitory computer-readable storage, may be used to perform at least portions of the herein described functionality.

Various aspects of the embodiments disclosed herein are performed on one or more computing devices, such as a laptop computer, tablet computer, mobile phone or other handheld computing device, or one or more servers. Such computing devices include processing circuitry embodied in one or more processors or logic chips, such as a central processing unit (CPU), graphics processing unit (GPU), field programmable gate array (FPGA), application-specific integrated circuit (ASIC), or programmable logic device (PLD). Further, the processing circuitry may be implemented as multiple processors cooperatively working in concert (e.g., in parallel) to perform the instructions of the inventive processes described above.

The process data and instructions used to perform various methods and algorithms derived herein may be stored in non-transitory (i.e., non-volatile) computer-readable medium or memory. The claimed advancements are not limited by the form of the computer-readable media on which the instructions of the inventive processes are stored. For example, the instructions may be stored on compact disks (CDs), digital versatile disks or digital video disks (DVDs), in FLASH memory, random access memory (RAM), read only memory (ROM), programmable read only memory (PROM), erasable programmable read only memory (EPROM), electrically erasable programmable read-only memory (EEPROM), hard disk or any other information processing device with which the computing device communicates, such as a server or computer. The processing circuitry and stored instructions may enable the computing device to perform, in some examples, the process flow 100 of FIG. 1A, the process flow 130 of FIG. 1B, the process flow 300 of FIG. 3, and/or the process flow 400 of FIG. 4.

These computer program instructions can direct a computing device or other programmable data processing apparatus to function in a particular manner, such that the instructions stored in the computer-readable medium produce an article of manufacture including instruction means which implement the function/operation specified in the illustrated process flows.

Embodiments of the present description rely on network communications. As can be appreciated, the network can be a public network, such as the Internet, or a private network such as a local area network (LAN) or wide area network (WAN) network, or any combination thereof and can also include public switched telephone network (PSTN) or Integrated Services Digital Network (ISDN) sub-networks. The network can also be wired, such as an Ethernet network, and/or can be wireless such as a cellular network including EDGE, 3G, 4G, and 5G wireless cellular systems. The wireless network can also include Wi-Fi®, Bluetooth®, Zigbee®, or another wireless form of communication. The network, for example, may support communications between the medical devices of the patient population 408 and the data store 406 and/or the machine learning engine 402 as described in relation to FIG. 4 or between the report generator 430 and the computing device having the display 432, as described in relation to FIG. 4. The network may support communications between the medical device controller 1000 and the data analytics system 1032 and/or the intermediate device 1034 as described in relation to FIG. 10.

The computing device further includes a display controller for interfacing with a display, such as a built-in display or LCD monitor. A general purpose input/output (I/O) interface of the computing device may interface with a keyboard, a hand-manipulated movement tracked I/O device (e.g., mouse, virtual reality glove, trackball, joystick, etc.), and/or touch screen panel or touch pad on or separate from the display. The display controller and display, for example, may enable presentation of the user interface generated by the report generator 430 of FIG. 4.

Moreover, the present disclosure is not limited to the specific circuit elements described herein, nor is the present disclosure limited to the specific sizing and classification of these elements. For example, the skilled artisan will appreciate that the circuitry described herein may be adapted based on changes on battery sizing and chemistry or based on the requirements of the intended back-up load to be powered.

The functions and features described herein may also be executed by various distributed components of a system. For example, one or more processors may execute these system functions, where the processors are distributed across multiple components communicating in a network. The distributed components may include one or more client and server machines, which may share processing, in addition to various human interface and communication devices (e.g., display monitors, smart phones, tablets, personal digital assistants (PDAs)). The network may be a private network, such as a LAN or WAN, or may be a public network, such as the Internet. Input to the system may be received via direct user input and received remotely either in real-time or as a batch process.

Although provided for context, in other implementations, methods and logic flows described herein may be performed on modules or hardware not identical to those described. Accordingly, other implementations are within the scope that may be claimed.

In some implementations, a cloud computing environment, such as Google Cloud Platform™ or Amazon Web Services™, may be used perform at least portions of methods or algorithms detailed above. The processes associated with the methods described herein can be executed on a computation processor, of a data center. The data center, for example, can also include an application processor that can be used as the interface with the systems described herein to receive data and output corresponding information. The cloud computing environment may also include one or more databases or other data storage, such as cloud storage and a query database. In some implementations, the cloud storage database, such as the Google Cloud Storage, may store processed and unprocessed data supplied by systems described herein. For example, the contents of the physiological raw data store 106, the processed data store 112, and/or the patient information store 116 of FIG. 1A and FIG. 1B, and/or the contents of the physiological raw data store 406, the processed data store 412, the patient information store 416, and/or the historic cardiac condition measures 426 of FIG. 4 may be maintained in a database structure.

The systems described herein may communicate with the cloud computing environment through a secure gateway. In some implementations, the secure gateway includes a database querying interface, such as the Google BigQuery platform. The data querying interface, for example, may support access by the ECG signal data classifier(s) 124a, 124b and/or the cardio-vibrational signal data classifier(s) 124c to the data repository 106, access by the machine learning engine 102 to the physiological raw data store 106, the processed data store 112, and/or the patient information data store 116 as described in relation to FIG. 1A and FIG. 1B. The data querying interface, in another example, may support access by the machine learning engine 402 to the physiological raw data store 406, the processed data store 412, and/or the patient information data store 416 as described in relation to FIG. 4. In a further example, the data querying interface may support access by the cardiac condition measure comparing engine 428 to the historic cardiac condition measures 426 as described in relation to FIG. 4. The data querying interface, in an additional example, may support access by the report generator 430 to the processed data sets 412 and/or the patient information data sets 416 as described in relation to FIG. 4.

All of the functionalities described in connection with one embodiment are intended to be applicable to the additional embodiments described below except where expressly stated or where the feature or function is incompatible with the additional embodiments. For example, where a given feature or function is expressly described in connection with one embodiment but not expressly mentioned in connection with an alternative embodiment, it should be understood that the inventors intend that that feature or function may be deployed, utilized or implemented in connection with the alternative embodiment unless the feature or function is incompatible with the alternative embodiment.

While certain embodiments have been described, these embodiments have been presented by way of example only, and are not intended to limit the scope of the present disclosures. Indeed, the novel methods, apparatuses and systems described herein can be embodied in a variety of other forms; furthermore, various omissions, substitutions and changes in the form of the methods, apparatuses and systems described herein can be made without departing from the spirit of the present disclosures. The accompanying claims and their equivalents are intended to cover such forms or modifications as would fall within the scope and spirit of the present disclosures.

1. A method for determining a cardiac condition measure of a patient wearing a wearable medical device based on machine learning analysis, the method comprising:
obtaining, by processing circuitry from the wearable medical device, physiological data representing a sample timeframe, the physiological data comprising
ECG data representing a plurality of ECG signals of a patient, wherein the plurality of ECG signals was collected by at least two ECG electrodes of the wearable medical device monitoring a heart of the patient, and/or
cardio-vibrational data representing a plurality of cardio-vibrational signals of the patient, wherein the plurality of cardio-vibrational signals was collected by at least one vibrational sensor of the wearable medical device monitoring the heart of the patient;
applying, by the processing circuitry, the physiological data to a machine learning engine to determine the cardiac condition measure of the patient, wherein applying comprises
applying the ECG data and/or the cardio-vibrational data to a first tier of the machine learning engine comprising one or more first machine learning classifiers to obtain a first result, and
applying the first result along with i) clinical information regarding the patient and/or ii) physiological metrics determined from signals collected by the wearable medical device to a second tier of the machine learning engine comprising one or more second machine learning classifiers to obtain a second result; and
determining, by the processing circuitry at least in part from the second result, the cardiac condition measure.

2. The method of claim 1, wherein the second tier comprises one or more fully connected artificial neural network layers.

3. The method of claim 1, wherein the first tier comprises one or more convolutional artificial neural network layers.

4. (canceled)

5. The method of claim 1, wherein the one or more first machine learning classifiers comprise an ECG trained machine learning classifier and a cardio-vibrational data trained machine learning classifier.

6.-8. (canceled)

9. The method of claim 1, wherein the cardiac condition measure is one of Class I heart failure (HF), Class II HF, Class III HF, or Class IV HF in accordance with the New York Heart Association (NYHA) classification system.

10. (canceled)

11. The method of claim 1, wherein the cardiac condition measure is an ejection fraction classification.

12. The method of claim 11, wherein the ejection fraction classification comprises one of:
a) an ejection fraction of greater than 30% as a first ejection fraction classification, and an ejection fraction of less than 30% as a second ejection fraction classification;
b) an ejection fraction of greater than 35% as the first ejection fraction classification, and an ejection fraction of less than 35% as the second ejection fraction classification;
c) an ejection fraction of greater than 40% as the first ejection fraction classification, and an ejection fraction of less than 40% as the second ejection fraction classification; or
d) an ejection fraction of greater than 45% as the first ejection fraction classification, and an ejection fraction of less than 45% as the second ejection fraction classification.

13. The method of claim 1, further comprising providing, by the processing circuitry, information regarding the cardiac condition measure to a remote computing device for review by a medical professional, clinician, or caregiver.

14. The method of claim 1, further comprising providing, by the processing circuitry in real time for review by the patient, a medical professional, clinician, or caregiver, information corresponding to the cardiac condition measure via an output device of the wearable medical device or a separate computing device.

15.-16. (canceled)

17. The method of claim 1, wherein the sample timeframe is between about 10 seconds to about 20 seconds, between about 20 seconds to about seconds, between about 30 seconds and about 40 seconds, between about 40 seconds and about 50 seconds, or between about 50 seconds and about 60 seconds.

18. The method of claim 1, wherein the physiological metrics comprise one or more of electromechanical activation time (EMAT), time-corrected EMAT (EMATc), left ventricular systolic time (LVST), S3 intensity, S4 intensity, S3 duration, S4 duration, systolic dysfunction index (SDI), or heart rate.

19. The method of claim 1, wherein the clinical information comprises one or more of an age of the patient, a gender of the patient, an indication of a coronary artery bypass grafting (CABG) procedure on the patient, an indication of a congenital heart defect (CONG) diagnosis of the patient, an indication of an implantable cardioverter defibrillator explant (EXPLANT) procedure on the patient, an indication of heart failure or cardiomyopathy (HFCM) in the patient, an indication of diagnosis of a prior myocardial infarction of the patient, an indication of prior ventricular fibrillation of the patient, an indication of prior ventricular tachycardia of the patient, or an indication of diagnosis of cardiac ischemia of the patient.

20.-22. (canceled)

23. The method of claim 1, wherein the physiological data representing the sample timeframe is obtained from a period of at least
a) one second prior to applying the physiological data to the machine learning engine,
b) one hour prior to applying the physiological data to the machine learning engine,
c) 24 hours to applying the physiological data to the machine learning engine, or
d) 3 days prior to applying the physiological data to the machine learning engine.

24.-26. (canceled)

27. The method of claim 1, further comprising comparing, by the processing circuitry, at least one historic cardiac condition measure of the patient to the cardiac condition measure to identify a change in cardiac condition.

28.-33. (canceled)

34. The method of claim 1, wherein an area under the curve (AUC) performance of the machine learning engine is at least 70%, between about 70% and about 75%, between about 75% and about 80%, or between about 80% and about 85%, or between about 85% and about 95%, or between about 95% and about 99% when classifying the approximation of the cardiac condition measure into at least two classifications.

35. A system for determining a cardiac condition measure of a patient wearing a wearable medical device based on machine learning analysis, the system comprising:
a non-volatile computer readable storage medium comprising physiological data representing a sample timeframe, the physiological data comprising
ECG data representing a plurality of ECG signals of the patient, wherein the plurality of ECG signals was collected by at least two ECG electrodes of the wearable medical device monitoring a heart of the patient, and/or
cardio-vibrational data representing a plurality of cardio-vibrational signals of the patient, wherein the plurality of cardio-vibrational signals was collected by at least one vibrational sensor of the wearable medical device monitoring the heart of the patient; and
a plurality of operations stored as a plurality of computer executable instructions to a non-transitory computer readable media and/or encoded in hardware logic, wherein the plurality of operations is configured to
apply the physiological data to a machine learning engine to determine the cardiac condition measure of the patient, wherein applying comprises
applying the ECG data and/or the cardio-vibrational data to a first tier of the machine learning engine comprising one or more first machine learning classifiers to obtain a first result, and
applying the first result along with i) clinical information regarding the patient and/or ii) physiological metrics determined from signals collected by the wearable medical device to a second tier of the machine learning engine comprising one or more second machine learning classifiers to obtain a second result; and
determine, at least in part from the second result, the cardiac condition measure.

36.-37. (canceled)

38. The system of claim 35, wherein i) the one or more first machine learning classifiers comprise at least two stages of machine learning classifiers of the first tier and/or ii) the one or more second machine learning classifiers comprise at least two stages of machine learning classifiers of the second tier.

39. (canceled)

40. The system of claim 35, wherein the one or more first machine learning classifiers are configured to implement sets of convolutional filters, each being sized between about 2 and 10 weights, between about 10 and 50 weights, between about 50 and 100 weights, or between about 100 and 10000 weights.

41.-47. (canceled)

48. The system of claim 35, wherein the plurality of operations is configured to provide, in real time for review by the patient, a medical professional, clinician, or caregiver, information corresponding to the cardiac condition measure via an output device of the wearable medical device or a separate computing device.

49. The system of claim 48, wherein providing the information corresponding to the cardiac condition measure comprises adding, for review within a medical professional patient information portal, a visual indication of worsened cardiac condition associated with the patient.

50. The system of claim 49, wherein the plurality of operations is configured to provide, for review within the medical professional patient information portal, a visual display representing at least a portion of the physiological metrics.

51.-133. (canceled)
